SEQUENCE LISTING (32 sequences)

SEQ ID NO: 1 - DNA, 57 bp, Homo sapiens
  source 1..57: mol_type = genomic DNA
agcaaggctcagcctcaagattcacagcatctcagacacagcctaggccgcaccagg

SEQ ID NO: 2 - DNA, 41 bp, Homo sapiens
  source 1..41: mol_type = genomic DNA
ctagcttttctcttctgtcaaccccacacgcctttggcaca

SEQ ID NO: 3 - DNA, 199 bp, Homo sapiens
  source 1..199: mol_type = genomic DNA
gcagttcggcggtcccgcgggtctgtctcttgcttcaacagtgtttggacggaacagatccggggactctcttccagcctccgaccgccctccgatttcctctccgcttgcaacctccgggaccatcttctcggccatctcctgcttctgggacctgccagcaccgtttttgtggttagctccttcttgccaaccaacc

SEQ ID NO: 4 - DNA, 137 bp, Homo sapiens
  source 1..137: mol_type = genomic DNA
attcccagcctcacatcactcacaccttgcatttcacccctgcatcccagtcgccctgcagcctcacacagatcctgcacacacccagacagctggcgctcacacattcaccgttggcctgcctctgttcaccctcc

SEQ ID NO: 5 - DNA, 51 bp, Homo sapiens
  source 1..51: mol_type = genomic DNA
gactctgggaggcgtttgggcccagagaagtggatccgccgcttgcgccgc

SEQ ID NO: 6 - DNA, 35 bp, Homo sapiens
  source 1..35: mol_type = genomic DNA
cctttccggcggtgacgacctacgcacacgagaac

SEQ ID NO: 7 - DNA, 42 bp, Homo sapiens
  source 1..42: mol_type = genomic DNA
acatatccactcctgctctccctcctgcaggtgaccccagcc

SEQ ID NO: 8 - DNA, 217 bp, Homo sapiens
  source 1..217: mol_type = genomic DNA
ggatgccgccccggacagtggcacctgggaagcctgggagtgtttgtcccatcggtagcttgaaataaacgctcccctcagacacccgctgggttctctgatgttattatggttgagatgcagctggtctctcctggttaattgacttcttattaccaacaagttacttgaaagagtgtcaccaataatcattaaagtacggcaggctgaaccttca

SEQ ID NO: 9 - DNA, 186 bp, Homo sapiens
  source 1..186: mol_type = genomic DNA
gatcacatttaaaagcatctcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatcg

SEQ ID NO: 10 - DNA, 144 bp, Homo sapiens
  source 1..144: mol_type = genomic DNA
gagccttctgagcccagcgacttctgaagggccccttgcaaagtaatagggcttctgcctaagcctctccctccagccaataggcagctttcttaactatcctaacaagccttggaccaaatggaaataaagctttttgatgca

SEQ ID NO: 11 - DNA, 249 bp, Homo sapiens
  source 1..249: mol_type = genomic DNA
gctatgaccgtgcagagggagcccggagtccgagtcaagcattgtgaattattacctaacctggggaaccgaggaccagaaggaaggaccaggcttccagctcctctgcaccagacctgaccagccaggacagggcctggggtgtgtgtgagtgtgagtgtgagcgagagggtgagtgtggtcagagtaaagctgctccacccccagattgcaatgctaccaataaagccgcctggtgtttacaactaa

SEQ ID NO: 12 - DNA, 401 bp, Homo sapiens
  source 1..401: mol_type = genomic DNA
tggctgcagcccctgcctgtgtgtctggtgttgaggggacgaagggacccctgcccctgtctgccagcagaggcagtagggcacagagggaagaggagggtggggccctggatgactttccagcctcttcaactgacttgctcccctctccttttcttctctttagagacccagcccagggccctcccacccttgcccagacctggtgggcccttcagagggaggggtggacctgtttctctttcactttcatttgctggtgtgagccatggggtgtgtatttgtatgtggggagtaggtgtttgaggttcccgttctttcccttcccaagtctctgggggtggaaaggaggaagagatactagttaaagattttaaaaatgtaaataaaatatacttccc

SEQ ID NO: 13 - DNA, 54 bp, Homo sapiens
  source 1..54: mol_type = genomic DNA
aagcactctgagtcaagatgagtgggaaaccatctcaataaacacattttggat

SEQ ID NO: 14 - DNA, 126 bp, Homo sapiens
  source 1..126: mol_type = genomic DNA
gcttcctagatagaaaccaaagcagtgcaagattcagttcaaggtcctgaaaaaagaaaaacattttactctgtgtaccttgtgtctttctaaatttctctctccaaaataaagttcaagcattaa

SEQ ID NO: 15 - DNA, 936 bp, synthetic construct
  source 1..936: mol_type = other DNA
atgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagaccttattggtatgggcaaatcaggcaaatctggtaatggttcttataggttacttgatcattacaaatatcttactgcatggtttgaacttcttaatttaccaaagaagatcatttttgtcggccatgattggggtgcttgtttggcatttcattatagccatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggacccaggattcttttccaatgctattgttgaaggtgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataa

SEQ ID NO: 16 - DNA, 19 bp, Bacteriophage T7
  source 1..19: mol_type = genomic DNA
taatacgactcactatagg

SEQ ID NO: 17 - DNA, 20 bp, synthetic construct
  source 1..20: mol_type = other DNA
ggccgcaacggagaccacgc

SEQ ID NO: 18 - DNA, 22 bp, synthetic construct
  source 1..22: mol_type = other DNA
gcgtggtctccgttgcggccgc

SEQ ID NO: 19 - DNA, 2223 bp, synthetic construct
  source 1..2223: mol_type = other DNA
gtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggatctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagcttgcaagcttcacgtgat

SEQ ID NO: 20 - DNA, 10 bp, synthetic construct
  source 1..10: mol_type = other DNA
gatcatcagt

SEQ ID NO: 21 - DNA, 23 bp, synthetic construct
  source 1..23: mol_type = other DNA
ggccgcaattctgacatgcggcc

SEQ ID NO: 22 - DNA, 25 bp, synthetic construct
  source 1..25: mol_type = other DNA
ggccgcaattctgacatgcggccgc

SEQ ID NO: 23 - DNA, 53 bp, Homo sapiens
  source 1..53: mol_type = genomic DNA
atgaagtgggtcacattcatcagcctgctgttcctgttcagcagcgcctacag

SEQ ID NO: 24 - DNA, 312 bp, synthetic construct
  source 1..312: mol_type = other DNA
cacggcccaaaggccacactgcaagatatcgtgctgcatctggaacctcaaaacgagatccctgtggacctgctgggccatggacagctgtctgattctgaggaagaaaacgacgagattgacggcgtgaaccaccagcacctgcctgccagaagagccgaaccacagcggcacaccatgctgtgcatgtgctgcaagtgtgaagccagaatcgagctggtggtcgagagctctgctgacgatctgcgggcttttcagcaactgttcctgaacaccctgagcttcgtgtgcccttggtgcgccagccagcag

SEQ ID NO: 25 - DNA, 290 bp, synthetic construct
  source 1..290: mol_type = other DNA
acggcgatacccctacactgcacgaatacatgctggatctgcagccggaaacaacagacctttatggctacggccagctgaacgactccagcgaagaggaagatgagatcgacggccccgctggccaggccgagcctgatagagcccactacaacatcgtgaccttctgctgcaagtgcgactctaccctgcggctgtgcgtgcagagcacccacgtggacatccggaccctggaggacctgctgatgggaacactgggcatcgtgtgccccatctgcagccagaagcct

SEQ ID NO: 26 - DNA, 185 bp, synthetic construct
  source 1..185: mol_type = other DNA
tccaggaccctcaggagcggcctcggaagctgcctcagctgtgcaccgagctgcagaccacaatccacgacatcatcctggagtgcgtgtactgtaaacagcagctgctcagacgcgaggtgtacgacttcgccttccgggatctgtgcattgtttaccgggacggcaacccttacgccgtgggc

SEQ ID NO: 27 - DNA, 141 bp, synthetic construct
  source 1..141: mol_type = other DNA
gacaagtgcctgaagttctacagcaagatcagcgagtaccggcactactgctacagcctgtacggtacgaccctggaacagcagtacaacaagcctctgtgtgacctcctcatcaggtgcatcaactgccagaaacctctg

SEQ ID NO: 28 - DNA, 108 bp, synthetic construct
  source 1..108: mol_type = other DNA
caacggcacctggacaaaaagcagagattccacaacatcagaggcagatggaccggcagatgtatgagctgctgtagaagctccagaacccggagagaaacccagctg

SEQ ID NO: 29 - DNA, 192 bp, synthetic construct
  source 1..192: mol_type = other DNA
gcccgttttgaggaccccaccagaagaccctacaagctgcccgacctgtgtacagagctgaataccagcctgcaggatatcgagatcacctgcgtgtactgtaaaaccgtgctggaactgaccgaggtgttcgagttcgccttcaaggacctgtttgtggtgtatcgggacagcatcccccacgccgccggc

SEQ ID NO: 30 - DNA, 140 bp, synthetic construct
  source 1..140: mol_type = other DNA
cataagtgcatcgacttctacagcagaatcagagagctgcgccactactccgatagcgtgtatggcgatacactggagaagctgaccaacacaggcctgtacaatctgctgatcaggtgtctgagatgccagaaacctct

SEQ ID NO: 31 - DNA, 126 bp, synthetic construct
  source 1..126: mol_type = other DNA
ctgagacacctcaacgagaagcggagatttcacaatatcgccggccactacagaggacagtgccactcttgttgtaatagagctagacaggagagactgcagagaagaagggaaacccaggtgtga

SEQ ID NO: 32 - DNA, 15 bp, synthetic construct
  source 1..15: mol_type = other DNA
ggcagcggctccggc

(19) United States
(12) Patent Application Publication
(10) Pub. No.: US 2026/0199446 A1
(43) Pub. Date: Jul. 16, 2026

(54) NUCLEIC ACID MOLECULE HAVING IMPROVED EXPRESSION EFFICIENCY BY MODIFICATION, EXPRESSION VECTOR AND PHARMACEUTICAL COMPOSITION INCLUDING THEREOF

(71) Applicant: SML BIOPHARM CO., LTD., Gyeonggi-do (KR)

(72) Inventors: Jae Hwan NAM, Gyeonggi-do (KR); Seong Hyun LEE, Gyeonggi-do (KR); Hye Won KWAK, Seoul (KR); Hyeong Jun PARK, Gyeonggi-do (KR); Ga Hyun ROH, Seoul (KR)

(73) Assignee: SML BIOPHARM CO., LTD., Gyeonggi-do, GG (KR)

(21) Appl. No.: 19/019,850

(22) Filed: Jan. 14, 2025

(51) Int. Cl. A61K 39/12; A61K 39/00; C12N 7/00; C12N 15/11; C12N 15/85

(52) U.S. Cl. CPC A61K 39/12; C12N 7/00; C12N 15/11; C12N 15/85; A61K 2039/53; A61K 2039/572; C12N 2310/531; C12N 2710/20034; C12N 2830/50

(57) ABSTRACT

Described herein are nucleic acid molecules that include a coding region encoding a peptide, or fragment thereof, and an element having a stem-loop structure located adjacently to the coding region, and the loop consists of a single type of nucleotide. The peptide, or fragment thereof, encoded in the coding region can be expressed stably and efficiency by the stem-loop structure.

### SEQUENCE LISTING

The instant application contains a Sequence Listing which has been submitted in ASCII format via EFS-Web and is hereby incorporated by reference in its entirety. Said ASCII copy, created on Jan. 7, 2025, is named Sequence Listing-SEQ.XXML.SUPP and is 34 kilobytes in size.

### BACKGROUND

### Technical Field

The present application is supported by Ministry of Science and Information and Communication Technology of Korean Government (New/Variant Infectious Disease Response Platform Core Technology Development Business; Platform Development for New mRNA Vaccines Project; Grant No. 171158916) and by Ministry of Food and Drug Safety of Korean Government (Infectious Disease Response Innovation Technology Support Research Business; Toxic Evaluation Technology Development Research of mRNA Vaccines Project; Grant No. 1475013322).

The present disclosure relates to a nucleic acid molecule, and more specifically, to a nucleic acid molecule with beneficial expression efficiency and stability, an expression vector or a composition for treating or preventing a disease including thereof, and a method of treating a disease by using the nucleic acid molecule

### Description of the Related Art

As biotechnology such as genetic recombination technology was developed in 1970s, various expression systems that express a gene of interest (GOI) have been known. Among the expression systems, cell-based expression systems typically uses natural expression mechanisms of micro-organisms or eukaryotes, while other expression systems generally use purified RNA polymerases, ribosome, tRNAs and ribonucleotides. In particular, proteins originated from eukaryotes perform post-translational modifications such as phosphorylation, methylation and glycosylation. Since micro-organisms do not have such post-translational modification mechanisms, eukaryotic expression systems have been used in case expressing eukaryotic originated proteins.

Eukaryotic expression systems may be utilized to a gene therapy in which GOI having an open reading frame (ORF) encoding a peptide or a protein for treating various diseases is inserted in the expression systems or to a genetic vaccine in which GOI having ORF encoding a peptide or a protein such as antigens is inserted in the expression systems. However, there remains a need to develop a nucleic acid expression system that can improve the expression efficiency of the GOI in the relevant arts.

### BRIEF SUMMARY

Accordingly, the present disclosure is directed to a nucleic acid molecule, an expression vector and pharmaceutical or medicinal applications that can reduce one or more of the problems due to the limitations and disadvantages of the related art.

An object of the present disclosure is to provide a nucleic acid expression platform or a nucleic acid expression system that can improve the expression efficiency and/or expression stability of the gene of interest.

Another object of the present disclosure is to provide a composition or a treating or preventing method utilizing the nucleic acid expression platform or the nucleic acid expression system.

In one aspect, the present disclosure provide a nucleic acid molecule that comprises a coding region encoding one or more peptides or a fragment thereof, and an element located adjacently to the coding region and having a stem-loop structure, wherein a loop in the stem-loop structure comprises a single type of nucleotides.

The nucleic acid molecule can further comprise at least one expression control element operably linked to the coding region.

The at least one expression control element can comprise at least one transcription control element and at least one translation control element.

The at least one translation control element can comprise an upstream translation control element located upstream of the coding region and a downstream translation control element located downstream of the coding region.

As an example, the downstream translation control element can comprise a first downstream translation control element and a second downstream translation control element located sequentially at a downstream of the coding region.

The at least one translation control element can comprise an element having a cap-dependent translation initiation activity.

Alternatively, the at least one translation control element can comprise a translation control element having an Internal Ribosomal Entry Site (IRES) activity.

The nucleic acid molecule can further comprise a polyadenylation signal sequence or a polyadenosine sequence located between the coding region and the element having the stem-loop structure, or located downstream of the element having the stem-loop structure.

The nucleic acid molecule can have an RNA type.

The loop of the stem-loop structure can consist of an adenosine or an analog thereof, or a nucleotide transcribed thereto.

A stem element located downstream of the element having the stem-loop structure can comprise at least one restriction recognition site or a fragment thereof.

The element having the stem-loop structure can further comprise a linker element interposed between a stem and the loop.

In one embodiment, the coding region can encode one or more therapeutic proteins or peptides.

The coding region can encode one or more peptides derived from human papilloma virus (HPV), a fragment thereof, or a tumor suppressive or attenuation peptide thereof.

As an example, the coding region can encode at least one of E6 peptide and E7 peptide of the human papilloma virus, a fragment thereof, or a tumor suppressive peptide thereof.

For example, the coding region can encode one or more peptides derived from at least one human papilloma virus (HPV) type of an HPV-16 type or an HPV-18 type, a fragment thereof, or a tumor suppressive or attenuation peptide thereof.

In another embodiment, the coding region can encode one or more reporter peptides, one or more marker or selection peptides, or a fragment thereof.

In another embodiment, the coding region can encode one or more immunogens, or a fragment thereof.

In another aspect, the present disclosure provides a recombinant expression vector including the nucleic acid molecule.

In another aspect, the present disclosure provides a method of treating or preventing a disease, the method comprising administering a pharmaceutically effective amount of the nucleic acid molecule of claim 1 or an expression construct including the nucleic acid molecule to a body.

In one or more embodiment, the nucleic acid expression platform includes an element having the stem-loop structure so that the platform can induce the gene of interest encoded in the coding region to be expressed stably. The final expression efficiency of the gene of interest can be improved by applying the nucleic acid molecule.

The expression efficiency of various genes of interests encoding such as a reporter protein, an infections antigen, a tumor antigen and/or a therapeutic peptide or protein can be improved by using the expression platform. The expression platform can be utilized in detecting or analyzing specific materials in a sample, or in preparing a genetic therapeutics or vaccines such as an mRNA vaccine and/or a protein subunit vaccine.

For example, the expression platform including nucleotides encoding a peptide derived from a human papilloma virus (HPV), a fragment thereof, or a tumor suppressive or attenuation peptide thereof in the coding region can be utilized to a composition or a method for treating or preventing a disease caused by HPV infections, for example, a cancer such as a cervical cancer.

### BRIEF DESCRIPTION OF THE SEVERAL VIEWS OF THE DRAWINGS

The accompanying drawings, which are included to provide a further understanding of the disclosure, are incorporated in and constitute a part of this specification, illustrate implementations of the disclosure and together with the description serve to explain the principles of embodiments of the disclosure.

Each of FIGS. 1 to 4 is a schematic diagram illustrating component of a nucleic acid molecule or a polynucleotide that can express a gene of interest efficiently in accordance with an exemplary embodiment of the present disclosure.

Each of FIGS. 5 to 10 is a graph illustrating the result of measuring the expression level of Renilla Luciferase at 6 hours and 24 hours, respectively, after transfecting Nor10 cells of HeLa cells with nucleic acid molecules having a coding region encoding Renilla Luciferase in accordance with Examples.

FIG. 11 illustrates the result of measuring the expression level of peptides in the nucleic acid molecule having a coding region encoding HPV E6 peptide and HPV E7 peptide in accordance with Examples.

Each of FIGS. 12 and 13 is a photograph or a graph illustrating the ELISPOT assay result or Flow cytometry assay result for evaluating the immune response after immunizing mice by injecting the nucleic acid molecules having the coding region encoding the HPV E6 peptide and HPV E7 peptide into the mice in accordance with Examples.

FIG. 14 is a graph illustrating a result of reducing tumor tissues after injecting tumor cell-lines into mice and injecting the nucleic acid molecule having the coding region encoding HPV E6 peptide and HPV E7 peptide into mice in accordance with Examples.

Each of FIGS. 15 to 17 is a graph illustrating Flow cytometry assay result for the immune cells in the mice spleen tissues after injecting tumor cell-lines into mice, injecting the nucleic acid molecule having the codding region encoding HPV E6 peptide and HPV E& peptide into mice, and isolating the mice spleen tissue in accordance with Examples.

Each of FIGS. 18 to 21 is a graph illustrating Flow cytometry assay result for the immune cells penetrating into the tumor cells after injecting tumor cell-lines into mice, injecting the nucleic acid molecule having the codding region encoding HPV E6 peptide and HPV E& peptide into mice, and isolating the mice spleen tissue in accordance with Examples.

### DETAILED DESCRIPTION

### Definitions

As used herein, the term “amino acid” is used in the broadest sense and is intended to include naturally occurring L-amino acids or residues thereof. Conventionally used one-character or three-character abbreviation for the naturally occurring amino acid is used herein. Amino acid includes not only D-amino acid but also chemically-modified amino acids, for example, amino acid analogs, naturally occurring amino acids that is not typically incorporated into proteins, for example, norleucine, and chemically-synthesized compounds with amino acid-like properties known to a relevant art. For example, phenylalanine or proline analogs or mimetics each of which permits conformational limitations as the same as natural phenylalanine Phe or proline Pro is included in the definition of amino acid. Such analogs and mimetics are referred as “functional equivalences” of amino acids herein. Other examples of amino acids are illustrated in documents (See [Roberts and Vellaccio, The peptides: Analysis, Synthesis, Biology, Eds. Gross and Meiehofer, Vol. 5, p. 341, Academic Press, Inc.: N.Y. 1983]).

For example, synthetic peptides by standard solid-phase synthesis technique are not limited to amino acids encoded by corresponding genes, and allows the given amino acids to be substituted with much widely various ranges. Amino acids that are not encoded by the genetic code are referred as “amino acid analog” herein. For example, amino acid analog includes, but are not limited to, 2-amino adipic acid (Aad) to Glu and Asp; 2-amino pimelic acid (Apm) to Glu and Asp; 2-amino butyric acid (Abu) to Met, Leu and other aliphatic amino acids; 2-amino heptanoic acid (Ahe) to Met, Leu and other aliphatic amino acids; 2-amino iso-butyric acid (Aib) to Gly; cyclohexyl alanine (Cha) to Val, Leu and Ile; homo arginine (Har) to Arg and Lys; 2,3-diamino propionic acid (Dap) to Arg and His; N-ethyl glycine (EtGly) to Gly, Pro and Ala; N-ethyl asparagine (EtAsn) to Asn and Gln; hydroxyl lysine (Hyl) to Lys; allo hydroxyl lysine (AHyl) to Lys; 3-(and 4-) hydroxyl proline (3Hyp, 4Hyp) to Pro, Ser and Thr; allo-isoleucine (AIle) to Ile, Leu and Val; 4-amidino phenyl alanine to Arg; N-methyl glycine (MeGly, sarcosine) to Gly, Pro and Ala; N-methyl isoleucine (MeIle) to Ile; norvaline (Nva) to Met and other aliphatic amino acids; ornithine (Orn) to Lys, Arg and His; citrulline (Cit) and methionine sulfoxide (MSO) to Thr, Asn and Gln; and N-methyl phenyl alanine (MePhe), trimethyl phenyl alanine, halo-(F—, Cl—, Br— or I—) phenyl alanine or trifluoryl phenyl alanine to Phe.

As used herein, the term ‘peptide’ includes any of proteins, fragments of the proteins and peptides that are isolated from naturally-occurring environment or synthesized by recombinant technique or chemical synthesis. For example, the peptides of the present disclosure may comprise, but is not limited to, at least 5, preferably 10 amino acids.

In an exemplary embodiment, compound variants, for example, peptide variants substituted with one or more amino acids are provided. As used herein, the term “peptide variants” includes modified peptides that have one or more substitutions, deletions, addition and/or insertions of amino acids and exhibit substantially the same biological functions as the original peptide. The peptide variants should have an identity of 70% or more, preferably 90% of more, more preferably 95% or more as the original peptide.

Such amino acid substituents may comprise, but is not limited to, “Conservative” amino acid substituents. Alternatively, the amino acid substituents may include non-conservative variants. In one exemplary embodiment, the polypeptide variants may have amino acid sequences different from an original amino acid sequence by substitutions, deletions, additions and/or insertions of 5 or less amino acids. In addition, peptide variants may be changed by deletions or additions of amino acids that have minimal effects upon immunogenicity, a secondary structure, and hydropathic nature of a peptide.

As used herein, the term “conservative” substitution means that there are little changes in the secondary structure and hydropathic nature of polypeptides in case amino acids of the polypeptides changed to other amino acids, unless mentioned otherwise. Such amino acid variations with regard to the conservative substitutions may be obtained based upon relative similarity of side chain substituents of amino acids, for example, polarity, charge, solubility, hydrophobicity, hydrophilicity, and/or amphipathic nature.

For example, amino acids may be divided as 1) hydrophobic (norleucine, methionine, alanine, valine, leucine, isoleucine), 2) neutral hydrophilic (cysteine, serine, threonine, asparagine, glutamine), 3) acidic (aspartic acid, glutamic acid), 4) basic (histidine, lysine, arginine), 5) residues having influence on the chain directions (glycine, proline), and 6) aromatic (tryptophan, tyrosine, phenylalanine) based upon the common side chains properties. Conservative variation will accompany an exchange of one member in each of the classes for another member in the same class.

It has been known that any of arginine, lysine and histidine has positively charged residue; alanine, glycine and serine has similar sizes; phenylalanine, tryptophan and tyrosine has similar shapes by analyzing the size, shapes and kinds the amino acids side chain substituents. Accordingly, each of arginine, lysine and histidine; each of alanine, glycine and serine; and each of phenylalanine, tryptophan and tyrosine may be biologically functional equivalents based upon those considerations.

Hydropathic index may be considered in introducing variations. Each amino acid is given hydropathic index based upon its own hydrophobicity and charge: Isoleucine (+4.5); Valine (+4.2); Leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamic acid (−3.5); glutamine (−3.5); aspartic acid (−3.5); asparagine (−3.5); lysine (−3.9); and arginine (−4.5).

Hydropathic index of amino acids is very important in bestowing peptides or proteins with interactive biological functions. It has been known that similar biological activities may be maintained in only substituting amino acids with other amino acids having similar hydropathic indices. In case of introducing variations considering the hydropathic index, reciprocal substitutions among amino acids having hydropathic index value differences within preferably ±2, more preferably ±1, further more preferably ±0.5 are done.

Also, it is well known that reciprocal substitutions among amino acids having similar hydrophilicity induce proteins having equivalently biological activities. As disclosed in U.S. Pat. No. 4,554,101, following hydrophilicity value are accorded to each amino acid residue: Arginine (+3.0); lysine (+3.0); aspartic acid (+3.0±1); glutamic acid (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In case of introducing variations considering the hydrophilicity value, reciprocal substitutions among amino acids having hydrophilicity value differences within preferably +2, more preferably +1, further more preferably +0.5 are done.

Amino acid exchanges in proteins that do not generally modify the molecular activities are known in the art (See, H. Neurath, R. L. Hill, The proteins, Academic Press, New York, 1979). The most commonly occurred exchanges are inter-exchange of amino acid residues between Ala/Ser, Val/Ile, Asp/Glu, Thr/Ser, Ala/Gly, Ala/Thr, Ser/Asn, Ala/Val, Ser/Gly, Thy/Phe, Ala/Pro, Lys/Arg, Asp/Asn, Leu/Ile, Leu/Val, Ala/Glu, and Asp/Gly.

Generally, the peptides (including fusion proteins) and polynucleotides described herein may be isolated. “Isolated” peptides or polynucleotides are separated from the original environment. For example, naturally occurring proteins are isolated by removing whole or part of co-existent material in a natural state. Such polypeptides should have purity of 90% or more, preferably 95% or more, more preferably 99% or more. Polynucleotides are isolated by cloning in the vectors.

As used herein, the term “polynucleotide” or “nucleic acid” are used inter-changeably, refers to polymers of any lengths of nucleotides, and includes comprehensibly DNA (i.e., cDNA) and RAN molecules. “Nucleotide”, which is a subunit of nucleic acid molecules, may comprises, but is not limited to, a deoxyribonucleotide, a ribonucleotide, a modified deoxyribonucleotide or a ribonucleotide, analogs thereof, and/or any substrates that can be incorporated into polynucleotides by DNA or RNA polymerase or synthetic reactions. Polynucleotide may comprise modified nucleotides, analogues having modified bases and/or polysaccharides such as methylated nucleotides and analogues thereof.

For example, an adenine (or adenosine) analog and/or an adenine (adenosine) derivative comprising an adenosine phosphate nucleotide may comprise, but is not limited to, 2′-O-methyladenosine, 7-deazaadenosine (tubercidin), 7-deaza-7-carbamolyadenosine (sangivamycin), 8-deazaadenosine, 2′-deoxyl-tubercidin, 4-amino-7-[3,4-dihydorxy-5-(hydroxymethyl)oxolan-2-yl]pyrido[2,3-d]pyrimidine-5-carbonitrile (toyocamycin), Purine-9-β-D-ribofuranoside; 9-β-D-Ribofuranosyl-9H-purine (ribosyl-isopurine, purinosine, nebularine), 2′-deoxyadenosine, 2′-deoxy-toyocamycin, 5′-N-ethylcarboxamidoadenosine and inosine.

A cytosine (or cytidine) analog and/or a cytosine (cytidine) derivative comprising a cytidine phosphate nucleotide may comprise, but is not limited to, fluorocytosine, deoxycytidine, cytosine arabinoside (cytarabine), 5-azacytidine, gemcitabine, 2′-2′-difluoro-deoxycytidine and 5-azacytosine arabinose.

A guanine (or guanosine) analog and/or a guanine (guanosine) derivative comprising a guanosine phosphate nucleotide may comprise, but is not limited to, hypoxanthine, mercaptopurine, thioguanine, 6-O-methyl guanosine, 7-thia-8-oxoguanosine, 7-deazaguanosine and 7,8-dihydro-8-oxo-7-(2-propen-1-yl)-guanosine (loxoribine).

A thymine (or thymidine) analog and/or a thymine (thymidine) derivative comprising a thymidine phosphate nucleotide, a uracil (or uridine) analog and/or a uracil (uridine) derivative comprising a uridine phosphate nucleotide may comprise, but is not limited to, azidothymidine (zidovudine), 5-trifluorothymidine (trifluridine), fluorouracil, 5-fluorouracil-2′-deoxyriboside (floxuridine), iodouridine, trifluridine, 4′-thio-idoxuridine and telbivudine.

In another embodiment, the nucleotide may comprise 5-modified cytidines and/or 5-modified uridines. The 5-modified cytidine may comprise, but is not limited to, 5-halocytidine (i.e., 5-iodocytidine, 5-bromocytidine), 5-alkinylcitydine, 5-heterocyclylcytidine, 5-hydroxycitidine, 5-carboxycitydine and/or 5-formylcytidine. The 5-modified uridine may comprise, but is not limited to, 5-halouridine (i.e., 5-iodouridine, 5-bromouiridine), 5-alkinyluridine, 5-heterocyclyluridine, 5-hydroxyuridine, 5-hydroxymethyluridine, 4-carboxyuridine and/or 5-formyluridine. Alternatively, 5′-modified uridine may be a nucleotide containing 2-deoxyribose.

A single type of nucleotides may comprise a naturally-synthesized nucleotide or a base, and the above analog or derivative or the modified nucleotides may be included in the single type of nucleotides.

Some variations in nucleotides do not result in variations of peptides or proteins. Such nucleic acid variants may include any nucleic acid molecules having codons encoding functionally equivalent or identical amino acids (for example, 6 codons encodes Arg or Ser by the degeneracy of codons) or encoding biologically equivalent amino acids. On the other hand, other variations in nucleotides may induce changes in peptides or proteins. In spite of variations causing changes of amino acids of proteins, it is possible to obtain variant proteins that show substantially the same activities as the proteins of the present disclosure.

A person having ordinary skill in the art will appreciate that peptides and nucleic acids herein is not limited to the peptides and the nucleic acids described in the Sequence Listing. Rather, it is intended that peptides or the proteins as well as the nucleic acid molecules encoding the peptides or the proteins of the present disclosure may comprise any amino acid sequences or nucleotide sequences that has substantially the same biological functions. For example, the biologically functional equivalent that can be included to a coding region operably linked to an expression control element and/or the recombinant protein/peptide expressed therefor may be a polynucleotide having variant base sequence exhibiting equivalent biological function as the coding region and/or a protein/peptide having variant amino acid sequence exhibiting equivalent biological function as the recombinant protein.

Considering the variations having the above-mentioned biological equivalent activity, the nucleic acid molecule encoding the peptide and/or protein according to the present disclosure is interpreted to also include sequences showing substantial identity with the sequences listed in the sequence listing. The above substantial identity is at least 61% when aligning the sequence of the present invention and any other sequence to correspond as much as possible and analyzing the aligned sequence using an algorithm commonly used in the art. It means a sequence showing homology, more preferably 70% homology, even more preferably 80% homology, and most preferably 90% homology. Alignment methods for sequence comparison are known in the art.

As used herein, the term “vector” means a construct or a vehicle that can be transfected or delivered into the host cells, and enables one or more genes of interest (or target genes of target sequences) to be expressed within the cells. In addition, a specific vector may indicate an expression of genes of open reading frames operably linked thereto. Such a vector may be referred to “recombinant expression vector” (or in short, “recombinant vector”) herein.

As used herein, the term “expression control/regulation sequence” or “expression control/regulation element” may mean nucleic acid sequences regulating or controlling transcriptional processes of the nucleic acid molecules and/or translational processes of the transcribed nucleic acid molecules. As used herein, the term “transcription control/regulation sequence” or “transcription control/regulation element” means that nucleic acid sequences regulating or controlling the transcriptional process of the nucleic acid molecules. For example, the transcription control element may comprise promoters such as a constitutive promoter or an inducible promoter, enhancers, and the likes.

In addition, as used herein, the term “translation control/regulation sequence” or “translation control/regulation element” means that nucleic acid sequences regulating or controlling translations processes of the transcript nucleic acid to a protein or a peptide. Each of the expression control sequence, the transcription control sequence and the translation control sequence is operatively linked to the target sequences to be expressed.

As used herein, the term “operably linked” means a functional linkage between expression control sequence such as promoters, signal sequences, ribosome binding sites and transcription terminal sequences and other nucleic acid sequences so that the expression control sequence may regulate transcriptions and/or translations of the other nucleic acid sequences.

### Nucleic Acid Molecule

The present disclosure relates to an expression platform improving expression efficiency and expression stability of a gene of interest by introducing a stem-loop structure. FIG. 1 is a schematic diagram illustrating components of a nucleic acid molecule in accordance with an exemplary embodiment of the present disclosure.

As illustrated in FIG. 1, a nucleic acid molecule NA1 comprises a coding region CR encoding one or more peptides or one or more proteins, and an element having a stem-loop structure (hereinafter, ‘stem-loop element’) SL1 located adjacently to the coding region CR. In addition, the nucleic acid molecule NA1 may further comprise one or more expression control element ECEs operably linked to the coding region CR and/or a polyadenylation signal sequence or a polyadenosine sequence PA that can be located downstream of the coding region CR.

If necessary, a cloning site including one or more restriction endonuclease recognition sites and/or one or more restriction endonuclease cut sites may be located upstream of the expression control element ECE, between the expression control element ECE and the coding region CR, between the coding region and the polyadenylation signal sequence or the polyadenosine sequence PA and/or a downstream of the polyadenylation signal sequence or the polyadenosine sequence PA or the stem-loop element SL1.

The stem-loop element SL1 may comprise a loop element L, a first stem element ST1 located upstream of the loop element L, and a second stem element ST2 located downstream of the loop element ST2. The first stem element ST1 may be reversely complementary (i.e., palindrome sequence), or at least partially reversely complementary to the second stem element ST2.

In one embodiment, the loop element L constituting a center of the stem-loop element SL1 may include about 5 to about 200, for example, about 10 to about 100, about 10 to about 50, about 10 to about 40, about 10 to about 30, or about 10 to about 20 identical nucleotides. As described above, the identical or same nucleotide comprises common adenosine nucleotides, cytidine nucleotides, guanosine nucleotides, thymidine nucleotides and uridine nucleotides, analogs thereof and/or derivatives thereof.

As an example, it is understood that the loop element L is composed of or consisting of adenosine nucleotides means that the loop element L is composed of only adenosine nucleotides, as well as adenosine nucleotides, analogs of adenine and/or adenosine, and/or derivatives of adenine and/or adenosine. The same meaning is applied to other nucleotides.

In one embodiment, the loop element L may consist of, but is not limited to, adenosine nucleotides, analogs of adenines and/or adenosines, derivatives of adenosines and/or adenosines, and/or nucleotides transcribed thereto.

At least of 60%, for example, at least 70%, at least 80% or at least 90% of the first stem element ST1 may be reversely complementary or partially reversely complementary to the second stem element ST2. Each of the first stem element ST1 and the second stem element ST2 may include, but is not limited to, 3 or more nucleotides, for example, about 5 to about 50 nucleotides, about 5 to about 30 nucleotides, about 5 to about 20 nucleotides, or about 5 to about 10 nucleotides.

In one embodiment, the stem-loop element SL1 may be located at the downstream of the polyadenylation signal sequence or the polyadenosine sequence PA. In this case, the second stem element ST2 located at the downstream of the loop element L constituting the stem-loop element SL1 may be or comprise one or more restriction endonuclease recognition sites and/or one or more restriction endonuclease cut sites, or a partial sequence thereof. The partial sequence of the restriction endonuclease recognition site or the restriction endonuclease cut sites may be about 50% or more, for example, about 60% or more, about 70% or more, about 80% or more, or about 90% or more of the restriction endonuclease recognition site or the restriction endonuclease cut site.

For example, the second stem element ST2 may be or comprise, or be a partial sequence thereof, but is not limited to, a site recognized by or cut by any restriction endonuclease selected from AngI, AatI, AbaI, BamHI, BbvI, BcgI, BplI, BsmAI, Alw26I, BsrI, ClaI, Earl, Eco57I, EcoRI, EcoRII, EcoRV, FokI, HaeIII, HindIII, HpaIII, HphI, KpnI, MboI, MluI, NaeI, NdeII, NgoMIV, NlaIII, NotI, PacI, PstI, SacI, SacII, SalI, SfaNI, SmaI, TaqI, XbaI, XhoI, PvuI, and combinations thereof, or partial sequences thereof. In this case, the first stem element ST1 would be or comprise sequences complement to the sites recognized by such restriction endonucleases, the site cut by the restriction endonucleases, or partial sequences thereof.

The coding region CR may consist or comprise one or more open reading frames (ORF) of a gene of interest (GOI). The kinds and the lengths of the ORF of the gene of interest inserted to the coding region CR are not limited.

In one embodiment, the coding region CR may encode one or more reporter peptides (reporter proteins) and/or one or more marker or selection peptides. For example, the reporter peptide may include, but is not limited to, luciferase, a green fluorescent protein (FGS), enhanced green fluorescent protein (EGFP), beta-galactosidase and combinations thereof. Alternatively or additionally, the marker or selection peptides may comprise, but is not limited to, alpha-globin, galactokinase, xanthine and combinations thereof. When the coding region CR includes one or more open reading frame encoding the reporter peptide and/or the marker or selection peptide, the nucleic acid molecule NA1 may be utilized as a composition for detecting or analyzing the presence or absence of specific material or molecule in a sample.

In another embodiment, the coding region CR may include one or more open reading frames encoding one or more peptides with an effect of treating or preventing diseases or a fragment thereof. The peptide with an effect of treating or preventing diseases of a fragment thereof may be a peptide associated with treatment or prevention of diseases or a fragment thereof in a pharmaceutical composition that will be described below. Alternatively, the peptide with an effect of treating or preventing diseases may comprise cytokines, lymphokines, monokines, growth factors, receptors, signaling molecules, transcription factor and/or apoptosis factors.

In one embodiment, the peptide with an effect of treating or preventing diseases, which can be encoded in the coding region CR, may comprise an adjuvant peptide. As an example, the adjuvant peptide may induce innate immune response. For example, pattern recognition receptors such as receptors selected from toll-like receptor superfamily including toll-like receptor (TLRs) that can be selected from human TLR1 to TLR10 or murine TLR1 to TRL13 can be included in such innate immune response. In another embodiment, the adjuvant peptide may comprise human adjuvant peptides, pathogenic adjuvant peptides (e.g., bacterial peptides, protozoan peptides, viral peptides, fungal peptides) and/or animal adjuvant peptides. If necessary, nucleotides encoding human peptides associated with adjuvant effect (e.g., ligands of pattern recognition receptors, pattern recognition receptors, proteins in the course of signaling, transcription factors and/or cytokines) can be inserted into the coding region CR.

In another embodiment, the coding region CR may encode nucleotides encoding one or more immunogens or a fragment thereof. As an example, the immunogen encoded in the coding region CR may comprise, but is not limited to, an antigen, an antigenic epitope, a fragment of an antigen, or a peptide of an antigen. As an example, the immunogen may comprise, but is not limited to, a protein or peptide antigen such as a tumor antigen, an allergic antigen or an allergen, an autoimmune auto-antigen, a pathogenic antigen. The pathogenic antigen encoded in the coding region CR may be antigens derived from viral antigens, bacterial antigens, fungal antigens, protozoan antigens, animal antigens and/or allergic antigens.

In one embodiment, the pathogenic antigen may be induced from pathogenic organisms causing immunological responses in individuals, for example, in mammalian individuals such as in a human body, for example, from bacterial, viral or protozoan (multi-cellular) pathogenic organisms. More particularly, the pathogenic antigen may comprise a surface antigen, for example, a protein (or a protein fragment, for example external part of the surface antigen) present on a surface of viral, bacterial or protozoan organisms.

As an example, the pathogenic antigen may comprise a peptide or protein antigen induced from pathogens associated with infectious diseases. For example, the pathogenic antigen may be induced from, but is not limited to, Acinetobacter baumannii, Anaplasma genus, Anaplasma phagocytophilum, Ancylostoma braziliense, Ancylostoma duodenale, Arcanobacterium haemolyticum, Ascaris lumbricoides, Aspergillus genus, Astroviridae, Babesia genus, Bacillus anthracis, Bacillus cereus, Bartonella henselae, BK virus, Blastocystis hominis, Blastomyces dermatitidis, Bordetella pertussis, Borrelia burgdorferi, Borrelia genus, Borrelia spp, Brucella genus, Brugia malayi, Bunyaviridae family, Burkholderia cepacia and other Burkholderia spp, Burkholderia mallei, Brukholderia psudomallei, Caliciviridae family, Campylobacter genus, Candida albicans, Candida spp, Chlamydia trachomatis, Chlamydophila pneumoniae, Chlamydophila psittaci, CJD prion, Clonorchis sinensis, Clostridium botulinum, Clostridium difficile, Clostridium perfringens, Clostridium perfringens, Clostridium spp, Clostridium tetani, Coccidioides spp, coronaviruses, Corynebacterium diphtheria, Coxiella burnetii, Crimean-Congo hemorrhagic fever virus, Cryptococcus neoformans, Cryptosporidium genus, Cytomegalovirus (CMV), Dengue viruses (DEN-1, DEN-2, DEN-3 and DEN-4), Dientamoeba fragilis, Ebolavirus (EBOV), Echinococcus genus, Ehrlichia chaffeensis, Ehrlichia ewingii, Ehrlichia genus, Entamoeba histolytica, Enterococcus genus, Enterovirus genus, Enteroviruses, particularly Coxsackie A virus) and EV71, Epidermophyton spp, Epstein-Barr Virus (EBV), Escherichia coli O157:H7, O111 and O104:H4, Fasciola hepatica and Fasciola gigantica, FFI prion, Filarioidea superfamily, Flaviviruses, Francisella tularensis, Fusobacterium genus, Geotrichum candidum, Giardia intestinalis, Gnathostoma spp, GSS prion, Guanarito virus, Haemophilus ducreyi, Haemophilus influenza, Helicobacter pylori, Henipavirus (Hendra virus Nipah virus), Hepatitis A virus, Hepatitis B virus (HPV), Hepatitis C virus (HCV), Hepatitis D virus, Hepatitis E virus, Herpes simplex viruses 1 and 2 (HSV-1 and HSV-2), Histoplasma capsulatum, HIV (Human immunodeficiency virus), Hortaea werneckii, Human bocavirus (HBoV), Human herpesvirus 6 (HHV-6) and Human herpesvirus 7 (HHV-7), Human metapneumovirus (hMPV), Human papillomavirus (HPV), Human parainfluenza viruses (HPIV), Japanese encephalitis virus, JC virus, Junin virus, Kingella kingae, Klebsiella granulomatis, Kuru prion, Lassa virus, Legionella pneumophila, Leishmania genus, Leptospira genus, Listeria monocytogenes, Lymphocytic choriomeningitis virus (LCMV), Machupo virus, Malassezia spp, Marburg virus, Measles virus, Metagonimus yokagawai, Microsporidia phylum, Molluscum contagiosum virus (MCV), Mumps virus, Mycobacterium leprae and Mycobacterium lepromatosis, Mycobacterium tuberculosis, Mycobacterium ulcerans, Mycoplasma pneumoniae, Naegleria fowleri, Necator americanus, Neisseria gonorrhoeae, Neisseria meningitides, Nocardia asteroids, Nocardia spp, Onchocerca volvulus, Orientia tsutsugamushi, Orthomyxoviridae family (influenza), Paracoccidioides brasiliensis, Paragonimus spp, Paragonimus westermani, Parvovirus B19, Pasteurella genus, Plasmodium genus, Pneumocystis jirovecii, Poliovirus, Rabies virus, Respiratory syncytial virus (RSV), Rhinovirus, Rickettsia akari, Rickettsia genus, Rickettsia prowazekii, Rickettsia rickettsia, Rickettsia typhi, Rift Valley fever virus, Rotavirus, Rubella virus, Sabia virus, Salmonella genus, Sarcoptes scabiei, SARS coronavirus, Schistosoma genus, Severe fever with thrombocytopenia syndrome virus (SFTSV), Shigella genus, Sin Nombre virus, Hantavirus, Sporothrix schenckii, Staphylococcus genus, Streptococcus agalactiae, Streptococcus pneumoniae, Streptococcus pyogenes, Strongyloides stercoralis, Taenia genus, Taenia solium, Tickborne encephalitis virus (TBEV), Toxocara canis or Toxocara cati, Toxoplasma gondii, Treponema pallidum, Trichinella spiralis, Trichomonas vaginalis, Trichophyton spp, Trichuris trichiura, Trypanosoma brucei, Trypanosoma cruzi, Ureaplasma urealyticum, Varicella zoster virus (VZV), Variola major or Variola minor), vCJD prion, Venezuelan equine encephalitis virus, Vibrio cholera, West Nile virus, Western equine encephalitis virus, Wuchereria bancrofti, Yellow fever virus, Yersinia enterocolitica, Yersinia pestis, and Yersinia pseudotuberculosis.

For example, the pathogenic antigen may comprise an antigen derived from pathogens selected from influenza viruses, respiratory syncytial virus (RSV), herpes simplex virus (HSV), human papilloma virus (HPV), human immunodeficiency virus (HIV), Plasmodium, Staphylococcus aureus, dengue virus, Chlamydia trachomatis, cytomegalovirus (CMV), hepatitis B virus (HBV), Mycobacterium tuberculosis, rabies virus, yellow fever virus, Middle East respiratory syndrome coronavirus Viruses (MERS-CoV), Zika virus and coronavirus.

In other embodiment, the coding region CR can comprise one or more open reading frames (ORFs) encoding a protein or a peptide of a tumor antigen or a fragment thereof, variants or derivatives thereof. The tumor antigen can be a melanocyte-specific antigen, testicular tumor antigen or a tumor-specific antigen, for example, CT-X antigen, non-X CT antigen, a binding partner for CT-X antigen or a binding partner for non-X CT antigen or a tumor-specific antigen, or a tumor-specific antigen, variants or derivatives of the tumor antigen.

The open reading frame encoding such tumor antigen may comprise, but is not limited to, a nucleotide sequence encoding 5T4, 707-AP, 9D7, AFP, AlbZIP HPG1, alpha-5-beta-1-integrin, alpha-5-beta-6-integrin, alpha-actini-4/m, alpha-methyl acyl-coenzyme A racemase, ART-4, ARTC1/m, B7H4, BAGE-1, BCL-2, bcr/abl, beta-catenin/m, BING-4, BRCA1/m, BRCA2/m, CA 15-3/CA 27-29, CA 19-9, CA72-4, CA125, calreticulin, CAMEL, CASP-8/m, cathepsin B, carhepsin L, CD19, CD20, CD22, CD25, CDE30, CD33, CD4, CD52, CD55, CD56, CD80, CDC27/m, CDK4/m, CDKN2A/m, CEA, CLCA2, CML28, CML66, COA-1/m, coactosin-like protein, collage XXIII, COX-2, CT-9/BRD6, Cten, cyclin B1, cyclin D1, cyp-B, CYPB1, DAM-10, DAM-6, DEKCAN, EFTUD2/m, EGFR, ELF2/m, EMMPRIN, EpCam, EphA2, EphA3, ErbB3, ETV6-AML1, EZH2, FGF-5, FN, Frau-1, G250, GAGE-1, GAGE-2, GAGE-3, GAGE-4, GAGE-5, GAGE-6, GAGE7b, GAGE-8, GDEP, GnT-V, gp100, GPC3, GPNMB/m, HAGE, HAST-2, hepsin, Her2/neu, HERV-K-MEL, HLA-A*0201-R17I, HLA-A11/m, HLA-A2/m, HNE, Homeobox NKX3.1, HOM-TES-14/SCP-1, HOM-TES-85, HPV-E6, HPV-E7, HSP70-2M, HST-2, hTERT, iCE, IGF-1R, IL-13Ra2, IL-2R, IL-5, immature laminin receptor, kallikrein-2, krein-4, Ki67, KIAA0205, KIAA0205/m, KK-LC-1, K-Ras/m, LAGE-A1, LDLR-FUT, MAGE-A1, MAGE-A2, MAGE-A3, MAGE-A4, MAGE-A6, MAGE-A9, MAGE-A10, MAGE-A12, MAGE-B1, MAGE-B2, MAGE-B3, MAGE-B4, MAGE-B5, MAGE-B6, MAGE-B10, MAGE-B16, MAGE-B17, MAGE-C1, MAGE-C2, MAGE-C3, MAGE-D1, MAGE-D2, MAGE-D4, MAGE-E1, MAGE-E2, MAGE-F1, MAGE-H1, MAGEL2, mammaglobin A, MART-1/melan-A, MART-2, MART-2/m, matrix protein 22, MC1R, M-CSF, MEi/m, mesothelin, MG50/PXDN, MMP11, MN/CA IX-antigen, MRP-3, MUC-1, MUC-2, MUM-1/m, MUM-2/m, MUM-3/m, myosin class I/m, NA88-A, N-acetylgluocosminyl transferase-V, Neo-PAP, Neo-PAP/m, NFYC/m, NGEP, NMP22, NPM/ALK, N-Ras/m, NSE, NY-ESO-1, NY-ESO-B, OA1, OFA-iLRP, OGT, OGT/m, OS-9, OS-9/m, Osteocalcin, Osteopontin, p15, p190 minor bcr-abl, p53, p53/m, PAGE-4, PAI-1, PAI-2, PAP, PART-1, PATE, PDEF, Pim-1-kinase, Pin-1, Pml/PAR alpha, POTE, PRAME, PRDX5/m, prostain, proteinase-3, PSA, PSCA, PSGR, PSM, PSMA, PTPRK/m, RAGE-1, RBAF600/m, RHAMM/CD168, RU1, RU2, 5-100, SAGE, SART-1, SART-2, SART-3, SCC, SIRT2/m, Sp17, SSX-1, SSX-2/HOM-MEL-40, SSX-4, STAMP-1, STEAP-1, Survivin, Survivin-2B, SYT-SSX-1, SYT-SSX-2, TA-90, TAG-72, TARP, TEL-AML1, TGF beta, TGF beta RII, TGM-4, TPI/m, TRAG-3, TRG, TRP-1, TRP-2/6b, TRP/INT2, TRP-p8, Tyrosinase, UPA, VEGFR1, VEGFR-2/FLK-1, WT1, immunoglobulin genotype of lymphoid blood cell or T-cell receptor genotype of lymphoid blood cell, or fragments, variants or derivatives thereof, or a transcript thereof.

In another exemplary embodiment, the coding region CR may comprise nucleotides encoding an antibody, or a fragment thereof. As an example, the antibody encoded in the coding region CR may comprise a naturally occurring antibody or an antibody expressed by a recombination. In one embodiment, the antibody may be an antibody proper for therapeutic and/or diagnostic purposes. For example, the antibody may comprise monoclonal antibodies, polyclonal antibodies and antibodies with plural epitope properties, and may comprise chimeric antibodies, human antibodies, humanized antibodies, bispecific antibodies, endomers, fragments or variants thereof, naturally occurring antibodies and antibodies produced by immunization in a host.

As an example, antibodies for the treatment of cancers or tumor diseases may comprise, but is not limited to, 1311-tositumomab (Follicular lymphoma, B cell lymphoma, leukemia), 3F8 (Neuroblastoma), 8H9, Abagovomab (Ovarian cancer), Adecatumumab (Prostate and breast cancer), Afutuzumab (Lymphoma), Alacizumab pegol, Alemtuzumab (B-cell chronic lymphocytic leukaemia, T-cell-Lymphoma), Amatuximab, AME-133v (Follicular lyrphoma, cancer), AMG 102 (Advanced Renal Cell Carcinoma), Anatumomab mafenatox (Non-small cell lung carcinoma), Apolizurmab (Solid Tumors, Leukemia, Non-Hodgkin-Lymphoma, Lymphoma), Bavituximab (Cancer, viral infections), Bectumomab (Non-Hodgkin's lymphoma), Belimumab (Non-Hodgkin lymphoma), Bevacizumab (Colon Cancer, Breast Cancer, Brain and Central Nervous System Tumors, Lung Cancer, f-lepatocellular Carcinoma, Kidney Cancer, Breast Cancer, Pancreatic Cancer, Bladder Cancer, Sarcoma, Melanoma, Esophageal Cancer; Stomach Cancer, Metastatic Renal Cell Carcinoma; Kidney Cancer, Glioblastoma, Liver Cancer, Proliferative Diabetic Retinopathy, Macular Degeneration), Bivatuzumab mertansine (Squamous cell carcinoma), Blinatumomab, Brentuximab vedotin (Hematologic cancers), Cantuzumab (Colon Cancer, Gastric Cancer, Pancreatic Cancer, NSCLC), Cantuzumab mertansine (Colorectal cancer), Cantuzumab ravtansine (Cancers) Capromab pendetide (Prostate cancer), Carlumab, Catumaxomab (Ovarian Cancer, Fallopian Tube Neoplasms, Peritoneal Neoplasms), Cetuximab (Metastatic colorectal cancer and head and neck cancer), Citatuzumab bogatox (Ovarian cancer and other solid tumors), Cixutumumatb (Solid tumors), Clivatuzurmab tetraxetan (Pancreatic cancer), CNTO 328 (B-Cell Non-Hodgkin's Lymphoma, Multiple Myeloma, Castleman's Disease, ovarian cancer), CNTO 95 (Melanoma), Conaturnurnab, Dacetuzumab (Hematologic cancers), Dalotuzumab, Denosumab (Myeloma, Giant Cell Tumor of Bone, Breast Cancer, Prostate Cancer, Osteoporosis), Detumomab (Lymphoma), Drozitumab, Ecromneximab (Malignant melanoma), Edrecolomab (Colorectal carcinoma), Elotuzumab (Multiple myeloma), Elsilimomab, Enavatuzumab, Ensituximab, Epratuzumab (Autoimmune diseases, Systemic Lupus Erythematosus, Non-Hodgkin-Lymphoma, Leukemia). Ertumaxomab (Breast cancer), Ertumaxomab (Breast Cancer), Etaracizumab (Melanoma, prostate cancer, ovarian cancer), Farletuzumab (Ovarian cancer), FBTA05 (Chronic lymphocytic leukaemia), Ficlatuzumab (Cancer), Figitumumab (Adrenocortical carcinoma, non-small cell lung carcinoma), Flanvotumab (Melanoma), Galiximnab (B-cell lymphoma), Galixinmab (Non-Hodgkin-Lymphoma), Ganitumab, GC1008 (Advanced Renal Cell Carcinoma; Malignant Melanoma, Pulmonary Fibrosis), Gemtuzumab (Leukemia), Gemtuzumab ozogamicin (Acute myelogenous leukemia), Girentuximab (Clear cell renal cell carcinoma), Glembatumurnab vedotin (Melanoma, breast cancer), GS6624 (Idiopathic pulmonary fibrosis and solid tumors), HuC242-DM4 (Colon Cancer, Gastric Cancer, Pancreatic Cancer), HuHMFTG1 (Breast Cancer), HuN901-DMI (Myeloma), Ibritumomab (Relapsed or refractory low-grade, follicular, or transformed B-cell non-Hodgkin's lymphorna (NHL), Icucumab, ID09C3 (Non-Hodgkin-Lymphoma), Indatuximab ravtansine, Inotuzumab ozogamicin, lntetumumab (Solid tumors (Prostate cancer, melanoma)), Ipilimumab (Sarcoma, Melanoma, Lung cancer, Ovarian Cancer leucemia, Lymphoma, Brain and Central Nervous System Tumors, Testicular Cancer, Prostate Cancer, Pancreatic Cancer, Breast Cancer), Iratumumab (Hodgkin's lymphoma), Labetuzumab (Colorectal cancer), Lexatumumab, Lintuzumab, Lorvotuzumab mertansine, Lucatumumab (Multiple myeloma, non-Hodgkin's lymphoma, Hodgkin's lymphoma), Lumiliximab (Chronic lymphocytic leukemia), Mapatumumab (Colon Cancer, Myeloma), Matuzumnab (Lung Cancer, Cervical Cancer, Esophageal Cancer), MDX-060 (Hodgkin-Lymphoma, Lymphoma), MEDI 522 (Solid Tumors, Leukemia, Lymphoma, Small Intestine Cancer, Melanoma), Mitumomab (Small cell lung carcinoma), Mogamulizumab, MORab-003 (Ovarian Cancer, Fallopian Tube Cancer, Peritoneal Cancer), MORab-009 (Pancreatic Cancer, Mesothelioma, Ovarian Cancer, Non-Small Cell Lung Cancer, Fallopian Tube Cancer, Peritoneal Cavity Cancer), Moxetumomab pasudotox, MT103 (Non-Hodgkin-Lymphoma), Nacolomab tafenatox (Colorectal cancer), Naptumomab estafenatox (Non-small cell lung carcinoma, renal cell carcinoma), Narnatumab, Necitumumab (Non-small cell lung carcinoma), Nimotuzumab (Squamous cell carcinoma, head and neck cancer, nasopharyngeal cancer, glioma), Ninotuzumab (Squamous cell carcinomas, Glioma, Solid Tumors, Lung Cancer), Olaratumab, Onartuzumab (Cancer), Oportuzumab monatox, Oregovomab (Ovarian cancer), Oregovomab (Ovarian Cancer, Fallopian Tube Cancer, Peritoneal Cavity Cancer), PAM4 (Pancreatic Cancer), Panitumumab (Colon Cancer, Lung Cancer, Breast Cancer; Bladder Cancer; Ovarian Cancer), Patritumab, Pemtumomab, Pertuzumab (Breast Cancer, Ovarian Cancer, Lung Cancer, Prostate Cancer), Pritumumab (Brain cancer), Racotumomab, Radretumab, Ramucirumab (Solid tumors), Rilotumumab (Solid tumors), Rituximab (Urticaria, Rheumatoid Arthritis, Ulcerative Colitis, Chronic Focal Encephalitis, Non-Hodgkin-Lyrnphoma, Lymphona, Chronic Lymphocytic Leukemia), Robatumumab, Samalizumab, SGN-30 (Hodgkin-Lymphoma, Lymphoma), SGN-40 (Non-Hodgkin-Lymphoma, Myeloma, Leukemia, Chronic Lymphocytic Leukemia), Sibrotuzumab, Siltuximab, Tabalumab (B-cell cancers), Tacatuzumab tetraxetan, Taplitumomab paptox, Tenatumomab, Teprotumumab (Hematologic tumors), TGN1412 (Chronic lymphocytic leukemia, rheumatoid arthritis), Ticilimumab (tremelimumab), Tigatuzumab, TNX-650 (Hodgkin's lymphoma), Tositumomab (Follicular lymphoma, B cell lymphornas, Leukemias, Myeloma), Trastuzumab (Breast Cancer, Endometrial Cancer, Solid Tumors), TRBS07 (Melanoma), Tremelimumab, TRU-0C16 (Chronic lymphocytic leukemia), TRU-016 (Non-Hodgkin lymphoma), Tucotuzunab celmoleukin, Ublituximab, Urelumab, Veltuzumab (Non-Hodgkin's lymphoma), Veltuzumab (IMIU-106) (Non-Hodgkin's lymphoma), Volociximab (Renal Cell Carcinoma, Pancreatic Cancer, Melanorna), Votumumab (Colorectal tumors), WX-G250 (Renal Cell Carcinoma), Zalutumumab (Head and Neck Cancer, Squamous Cell Cancer), and Zanolimumab (T-Cell-Lyrnphoma).

Antibodies for the treatment of immune disorders may comprise, but is not limited to, Efalizumab (Psoriasis), Epratuzumab (Autoimmune diseases, Systemic Lupus Erythematosus, Non-Hodgkin-Lymphona, Leukemia), Etrolizumab (inflammatory bowel disease), Fontolizumab (Crohn's disease), Ixekizumab (autoimmune diseases), Mepolizumab (Hypereosinophilie-Syndrom, Asthma, Eosinophilic Gastroenteritis, Churg-Strauss Syndrome, Eosinophilic Esophagitis), Milatuzumab (multiple myeloma and other hematological malignancies), pooled immunoglobulins (Primary immunodeficiencies), Priliximab (Crohn's disease, multiple sclerosis), Rituximab (Urticaria, Rheumatoid Arthritis, Ulcerative Colitis, Chronic Focal Encephalitis, Non-Hodgkin-Lymphoma, Lymphoma, Chronic Lymphocytic Leukemia), Rontalizumab (systemic lupus erythematosus), Ruplizumab (rheumatic diseases), Sarilumab (rheumatoid arthritis, ankylosing spondylitis), Vedolizumab (Crohn's disease, ulcerative colitis), Visilizumab (Crohn's disease, ulcerative colitis), Reslizumab (inflammations of the airways, skin and gastrointestinal tract), Adalimumab (Rheumatoid arthritis, Crohn's disease, Ankylosing spondylitis, Psoriatic arthritis), Aselizumab (severely injured patients), Atinumab (treatment of neurologic systems), Atlizumab (rheumatoid arthritis, systemic juvenile idiopathic arthritis), Bertilimumab (severe allergic disorders), Besilesomab (inflammatory lesions and metastases), BIMS-945429, ALD518 (cancer and rheumatoid arthritis), Briakinumab (psoriasis, rheumatoid arthritis, inflammatory bowel diseases, multiple sclerosis), Brodalumab (inflammatory diseases), Canakinumab (rheumatoid arthritis), Canakinumab (cryopyrin-associated periodic syndromes (CAPS), rheumatoid arthritis, chronic obstructive pulmonary disease), Certolizumab pegol (Crohn's disease), Erlizumab (heart attack, stroke, traumatic shock), Fezakinumab (rheumatoid arthritis, psoriasis), Golimumab (rheumatoid arthritis, psoriatic arthritis, ankylosing spondylitis), Gomiliximab (allergic asthma), Infliximab (Rheumatoid arthritis, Crohn's disease, ankylosing spondylitis, psoriatic arthritis, plaque psoriasis, Morbus Bechterew, Colitis ulcerosa), Mavrilimumab (rheumatoid arthritis), Natalizumab (Multiple sclerosis), Ocrelizumab (multiple sclerosis, rheumatoid arthritis, lupus erythematosus, hematological cancer), Odulimomab (prevention of organ transplant rejections, immunological diseases), Ofatumumab (Chronic lymphocytic leukemia, follicular non-Hodgkin's lymphoma, B cell lymphoma, rheumatoid arthritis, relapsing remitting multiple sclerosis, Lymphoma, B-Cell Chronic Lymphocytic Leukemia), Ozoralizumab (inflammation), Pexelizumab (reduction of side effects of cardiac surgery), Rovelizumab (haemorrhagic shock), SBI-087 (Rheumatoid arthritis), SBI-087 (Systemic lupus erythematosus), Secukinunnab (uveitis, rheumatoid arthritis psoriasis), Sirukumab (rheumatoid arthritis), Talizumab (allergic reaction), Tocilizumab (rheumatoid arthritis, systemic juvenile idiopathic arthritis, Castleman's disease), Toralizumab (rheumatoid arthritis, lupus nephritis), TRU-015 (Rheumatoid arthritis), TRU-016 (Autoimmune disease and inflammation), Ustekinumab (multiple sclerosis, psoriasis, psoriatic arthritis), Ustekinumab (IL-12/IL-23 blocker) (Plaque-Psoriasis, psoriatic arthritis, multiple sclerosis, sarcoidosis, the latter versus), Vepalimomab (inflammation), Zolimomab aritox (systemic lupus erythematosus, graft-versus-host disease), Sifalimumab (SLE, dermatomyositis, polymyositis), Lumiliximab (Allergies), and Rho(D) Immune Globulin (Rhesus disease).

Antibodies for the treatment of infectious diseases may comprise, but is not limited to, Afelimomab (sepsis), CR6261 (infectious disease/influenza A), Edobacomab (sepsis caused by gram-negative bacteria), Efungumab (invasive Candida infection), Exbivirumab (hepatitis B), Felvizumab (respiratory syncytial virus infection), Foravirumab (rabies (prophylaxis)), Ibalizumab (HIV infection), Libivirumab (hepatitis B), Motavizumab (respiratory syncytial virus (prevention)), Nebacumab (sepsis), Tuvirumab (chronic hepatitis B), Urtoxazumab (diarrhoea caused by E. coli), Bavituximab (diverse viral infections), Pagibaximab (sepsis (e.g. Staphylococcus)), Palivizumab (prevention of respiratory syncytial virus infection in high-risk paediatric patients), Panobacumab (Pseudomonas aeruginosa infection), PRO140 (HIV infection), Rafivirumab (rabies (prophylaxis)), Raxibacumab (anthrax (prophylaxis and treatment)), Regavirumab (cytomegalovirus infection), Sevirumab (cytomegalovirus infection), Suvizumab (viral infections), and Tefibazumab (Staphylococcus aureus infection).

Antibodies for the treatment of blood disorders may comprise, but is not limited to. Abeiximab (percutaneous coronary intervention), Atorolimumab (hemolytic disease of the newborn), Eculizumab (Paroxysmal nocturnal haemoglobinuria), Mepolizumab (Hypereosinophilie-Syndrom, Asthma, Eosinophilic Gastroenteritis, Churg-Strauss Syndrome, Eosinophilic Esophagitis), and Milatuzumab (multiple myeloma and other hematological malignancies).

Antibodies used for immuno-regulation may comprise, but is not limited to, Antithymocyte globulin (Acute kidney transplant rejection, aplastic anaemia), Basiliximab (Prophylaxis against allograft rejection in renal transplant patients receiving an immunosuppressive regimen including cyclosporine and corticosteroids), Cedelizunab (prevention of organ transplant rejections, treatment of autoimmune diseases), Daclizumab (Prophylaxis against acute allograft rejection in patients receiving renal transplants, Multiple Sclerosis), Gavilimomab (graft versus host disease), Inolimomab (graft versus host disease), 1 Muromonab-CD3 (prevention of organ transplant rejections), Muromonab-CD3 (Acute renal allograft rejection or steroid-resistant cardiac or hepatic allograft rejection), Odulimomab (prevention of organ transplant rejections, immunological diseases), and Siplizumab (psoriasis, graft-versus-host disease (prevention)).

Antibodies used for the treatment of diabetes may comprise, but is not limited to, Gevokizumab (diabetes), Otelixizumab (diabetes mellitus type 1), and Teplizumab (diabetes mellitus type 1). Antibodies used for the treatment of Alzheimer's disease may comprise, but is not limited to, Bapineuzumab, Crenezunab, Gantenerumab, Ponezumab, R1450, and Solanezumab. Antibodies used for the treatment of asthma may comprise, but is not limited to, Benralizurnab, Enokizurnab, Keliximab, Lebrikizumab, Omalizumab, Oxelumab, Pascolizumab, and Tralokinumab.

In addition, antibodies which are used for the treatment of diverse disorders may comprise, but is not limited to, Blosozumab (osteoporosis), CaroRx (Tooth decay), Fresolimumab (idiopathic pulmonary fibrosis, focal segmental glomerulosclerosis, cancer), Fulranumab (pain), Romosozumab (osteoporosis), Stamulumab (muscular dystrophy), Tanezumab (pain), and Ranibizurnab (Neovascular age-related macular degeneration).

In another embodiment, the coding region CR may comprise one or more open reading frames encoding therapeutic or preventive proteins/peptides, a fragment thereof, variants or derivative thereof.

For example, the peptides or proteins used for treating metabolic or endocrine disorders (specific disease for therapeutic protein) may comprise, but is not limited to, Acid sphingomyelinase (Niemann-Pick disease), Adipotide (obesity), Agalsidase-beta (human galactosidase A) (Fabry disease; prevents accumulation of lipids that could lead to renal and cardiovascular complications), Alglucosidase (Pompe disease (glycogen storage disease type II)), alpha-galactosidase A (alpha-GAL A, Agalsidase alpha) (Fabry disease), alpha-glucosidase (Glycogen storage disease (GSD), Morbus Pompe), alpha-L-iduronidase (mucopolysaccharidoses (TMPS), Hurler syndrome, Scheie syndrome), alpha-N-acetylglucosaminidase (Santilippo syndrome), Amphiregulin (cancer, metabolic disorder), Angiopoietin ((Ang1, Ang2, Ang3, Ang4, ANGPTL2, ANGPTL3, ANGPTL4, ANGPTL5, ANGPTL6, ANGPTL7) (angiogenesis, stabilize vessels), Betacellulin (metabolic disorder), Beta-glucuronidase (Sly syndrome), Bone morphogenetic protein BMPs (BMP1, BMP2, BMP3, BMP4, BMP5, BMP6, BMP7, BMP8a, BMP8b, BMP10, BMP15) (regenerative effect, bone-related conditions, chronic kidney disease (CKD)), CLN6 protein (CLN6 disease—Atypical Late Infantile, Late Onset variant, Early Juvenile, Neuronal Ceroid Lipofuscinoses (NCL)), Epidermal growth factor (EGF) (wound healing, regulation of cell growth, proliferation, and differentiation), Epigen (metabolic disorder), Epiregulin (metabolic disorder), Fibroblast Growth Factor (FGF, FGF-1, FGF-2, FGF-3, FGF-4, FGF-5, FGF-6, FGF-7, FGF-8, FGF-9, FGF-10, FGF-11, FGF-12, FGF-13, FGF-14, FGF-16, FGF-17, FGF-17, FGF-18, FGF-19, FGF-20, FGF-21, FGF-22, FGF-23) (wound healing, angiogenesis, endocrine disorders, tissue regeneration), Galsulphase (Mucopolysaccharidosis VI), Ghrelin (irritable bowel syndrome (IBS), obesity, Prader-Willi syndrome, type II diabetes mellitus), Glucocerebrosidase (Gaucher's disease), GM-CSF (regenerative effect, production of white blood cells, cancer), Heparin-binding EGF-like growth factor (HB-EGF) (wound healing, cardiac hypertrophy and heart development and function), Hepatocyte growth factor HGF (regenerative effect, wound healing), Hepcidin (iron metabolism disorders, Beta-thalassemia), Human albumin (Decreased production of albumin (hypoproteinaemia), increased loss of albumin (nephrotic syndrome), hypovolaemia, hyperbilirubinaemia), Idursulphase (Iduronate-2-sulphatase) (Mucopolysaccharidosis II (Hunter syndrome)), Integrins αVβ3, αVβ5 and β5β11 (Bind matrix macromolecules and proteinases, angiogenesis), Iuduronate sulfatase (Hunter syndrome), Laronidase (Hurler and Hurler-Scheie forms of mucopolysaccharidosis I), N-acetylgalactosamine-4-sulfatase (rhASB; galsulfase, Arylsulfatase A (ARSA), Arylsulfatase B (ARSB)) (arylsulfatase B deficiency, Maroteaux-Lamy syndrome, mucopolysaccharidosis VI), N-acetylglucosamine-6-sulfatase (Sanfilippo syndrome), Nerve growth factor (NGF, Brain-Derived Neurotrophic Factor (BDNF), Neurotrophin-3 (NT-3), and Neurotrophin 4/5 (NT-4/5) (regenerative effect, cardiovascular diseases, coronary atherosclerosis, obesity, type 2 diabetes, metabolic syndrome, acute coronary syndromes, dementia, depression, schizophrenia, autism, Rett syndrome, anorexia nervosa, bulimia nervosa, wound healing, skin ulcers, corneal ulcers, Alzheimer's disease), Neuregulin (NRG1, NRG2, NRG3, NRG4) (metabolic disorder, schizophrenia), Neuropilin (NRP-1, NRP-2) (angiogenesis, axon guidance, cell survival, migration), Obestatin (irritable bowel syndrome (IBS), obesity, Prader-Willi syndrome, type II diabetes mellitus), Platelet Derived Growth factor (PDGF (PDFF-A, PDGF-B, PDGF-C, PDGF-D) (regenerative effect, wound healing, disorder in angiogenesis, Arteriosclerosis, Fibrosis, cancer), TGF beta receptors (endoglin, TGF-beta I receptor, TGF-beta 2 receptor, TGF-beta 3 receptor) (renal fibrosis, kidney disease, diabetes, ultimately end-stage renal disease (ESRD), angiogenesis), Thrombopoietin (THPO) (Megakaryocyte growth and development factor (MGDF)) (platelets disorders, platelets for donation, recovery of platelet counts after myelosuppressive chemotherapy), Transforming Growth factor (TGF; (TGF-a, TGF-beta (TGF-beta1, TGF-beta2, and TGF-beta3)) (regenerative effect, wound healing, immunity, cancer, heart disease, diabetes, Marfan syndrome, Loeys-Dietz syndrome), VEGF (VEGF-A, VEGF-B, VEGF-C, VEGF-D, VEGF-E, VEGF-F and PIGF) (regenerative effect, angiogenesis, wound healing, cancer, permeability), Nesiritide (Acute decompensated congestive heart failure), Trypsin (Decubitus ulcer, varicose ulcer, debridement of eschar, dehiscent wound, sunburn, meconium ileus), adrenocorticotrophic hormone (ACTI-L) (“Addison's disease, Small cell carcinoma, Adrenoleukodystrophy, Congenital adrenal hyperplasia, Cushing's syndrome, Nelson's syndrome, Infantile spasms), Atrial-natriuretic peptide (ANP) (endocrine disorders), Cholecystokinin (diverse), Gastrin (hypogastrinemia), Leptin (Diabetes, hypertriglyceridemia, obesity), Oxytocin (stimulate breastfeeding, non-progression of parturition), Somatostatin (symptomatic treatment of carcinoid syndrome, acute variceal bleeding, and acromegaly, polycystic diseases of the liver and kidney, acromegaly and symptoms caused by neuroendocrine tumors), Vasopressin (antidiuretic hormone) (diabetes insipidus), Calcitonin (Postmenopausal osteoporosis, Hypercalcaemia, Paget's disease, Bone metastases, Phantom limb pain, Spinal Stenosis), Exenatide (Type 2 diabetes resistant to treatment with netformin and a sulphonylurea), Growth hormone (GH), somatotropin (Growth failure due to GH deficiency or chronic renal insufficiency, Prader-Willi syndrome, Turner syndrome, AIDS wasting or cachexia with antiviral therapy), Insulin (Diabetes mellitus, diabetic ketoacidosis, hyperkalaemia), Insulin-like growth factor I IGF-1 (Growth failure in children with GH gene deletion or severe primary IGF1 deficiency, neurodegenerative disease, cardiovascular diseases, heart failure), Mecasermin rinfabate, IGF-1 analog (Growth failure in children with GH gene deletion or severe primary IGF1 deficiency, neurodegenerative disease, cardiovascular diseases, heart failure), Mecasermin, IGF-1 analog (Growth failure in children with GH gene deletion or severe primary IGF1 deficiency, neurodegenerative disease, cardiovascular diseases, heart failure), Pegvisomant (Acromegaly), Pramlintide (Diabetes mellitus, in combination with insulin), Teriparatide (human parathyroid hormone residues 1-34) (Severe osteoporosis), Becaplemin (Debridement adjunct for diabetic ulcers), Dibotermin-alpha (Bone morphogenetic protein 2) (Spinal fusion surgery, bone injury repair), Histrelin acetate (gonadotropin releasing hormone; GnRH) (Precocious puberty), Octreotide (Acromegaly, symptomatic relief of VIP-secreting adenoma and metastatic carcinoid tumours), and Palifermin (keratinocyte growth factor; KGF) (Severe oral mucositis in patients undergoing chemotherapy, wound healing). (in brackets is the particular disease for which the therapeutic protein is used in the treatment).

The therapeutic the protein (specific disease for protein treatment) with regard to the treatment of blood disorders, diseases of the circulatory system, diseases of the respiratory system, cancer or tumor diseases, infectious diseases or immune-deficiencies may comprise, but is not limited to, Alteplase (tissue plasminogen activator; tPA) (Pulmonary embolism, myocardial infarction, acute ischaemic stroke, occlusion of central venous access devices), Anistreplase (Thrombolysis), Antithrombin III (AT-III) (Hereditary AT-III deficiency, Thromboembolism), Bivalirudin (Reduce blood-clotting risk in coronary angioplasty and heparin-induced thrombocytopenia), Darbepoetin-alpha (Treatment of anaemia in patients with chronic renal insufficiency and chronic renal failure (+/−dialysis)), Drotrecogin-alpha (activated protein C) (Severe sepsis with a high risk of death), Erythropoietin, Epoetin-alpha, erythropoietin, erythropoietin (Anaemia of chronic disease, myelodysplasia, anaemia due to renal failure or chemotherapy, preoperative preparation), Factor IX (Haemophilia B), Factor VIIa (Haemorrhage in patients with haemophilia A or B and inhibitors to factor VIII or factor IX), Factor VIII (Haemophilia A), Lepirudin (Heparin-induced thrombocytopenia), Protein C concentrate (Venous thrombosis, Purpura fulminans), Reteplase (deletion mutein of tPA) (Management of acute myocardial infarction, improvement of ventricular function), Streptokinase (Acute evolving transmural myocardial infarction, pulmonary embolism, deep vein thrombosis, arterial thrombosis or embolism, occlusion of arteriovenous cannula), Tenecteplase (Acute myocardial infarction), Urokinase (Pulmonary embolism), Angiostatin (Cancer), Anti-CD22 immunotoxin (Relapsed CD33⁺ acute myeloid leukaemia), Denileukin diftitox (Cutaneous T-cell lymphoma (CTCL)), Immunocyanin (bladder and prostate cancer), MPS (Metallopanstimulin) (Cancer), Aflibercept (Non-small cell lung cancer (NSCLC), metastatic colorectal cancer (mCRC), hormone-refractory metastatic prostate cancer, wet macular degeneration), Endostatin (Cancer, inflammatory diseases like rheumatoid arthritis as well as Crohn's disease, diabetic retinopathy, psoriasis, and endometriosis), Collagenase (Debridement of chronic dermal ulcers and severely burned areas, Dupuytren's contracture, Peyronie's disease), Human deoxy-ribonuclease I, dornase (Cystic fibrosis; decreases respiratory tract infections in selected patients with FVC greater than 40% of predicted), Hyaluronidase (Used as an adjuvant to increase the absorption and dispersion of injected drugs, particularly anaesthetics in ophthalmic surgery and certain imaging agents), Papain (Debridement of necrotic tissue or liquefication of slough in acute and chronic lesions, such as pressure ulcers, varicose and diabetic ulcers, burns, postoperative wounds, pilonidal cyst wounds, carbuncles, and other wounds), L-Asparaginase (Acute lymphocytic leukaemia, which requires exogenous asparagine for proliferation), Peg-asparaginase (Acute lymphocytic leukaemia, which requires exogenous asparagine for proliferation), Rasburicase (Paediatric patients with leukaemia, lymphorna, and solid tumours who are undergoing anticancer therapy that may cause tumour lysis syndrome), Human chorionic gonadotropin (HCG) (Assisted reproduction), Human follicle-stimulating hormone (FSH) (Assisted reproduction), Lutropin-alpha (Infertility with luteinizing hormone deficiency), Prolactin (Hyperprolactinemia, serum prolactin deficiency, ovarian dysfunction in women, anxiety, arteriogenic erectile dysfunction, premature ejaculation, oligozoospermia, asthenospermia, hypofunction of seminal vesicles, hypoandrogenism in men), alpha-1-Proteinase inhibitor (Congenital antitrypsin deficiency), Lactase (Gas, bloating, cramps and diarrhea due to inability to digest lactose), Pancreatic enzymes (lipase, amylase, protease) (Cystic fibrosis, chronic pancreatitis, pancreatic insufficiency, post-Billroth II gastric bypass surgery, pancreatic duct obstruction, steatorrhoea, poor digestion, gas, bloating), Adenosine deaminase (pegademase bovine, PEG-ADA) (Severe combined immunodeficiency disease due to adenosine deaminase deficiency), Abatacept (Rheumatoid arthritis (especially when refractory to TNFa inhibition)), Alefacept (Plaque Psoriasis), Anakinra (Rheumatoid arthritis), Etanercept (Rheumatoid arthritis, polyarticular-course juvenile rheumatoid arthritis, psoriatic arthritis, ankylosing spondylitis, plaque psoriasis, ankylosing spondylitis), Interleukin-1 (IL-1) receptor antagonist, Anakinra (inflammation and cartilage degradation associated with rheumatoid arthritis), Thymulin (neurodegenerative diseases, rheumatism, anorexia nervosa), TNF-alpha antagonist (autoimmune disorders such as rheumatoid arthritis, ankylosing spondylitis, Crohns disease, psoriasis, hidradenitis suppurativa, refractory asthma), Enfuvirtide (HIV-1 infection), and Thymosin al (Hepatitis B and C).

For example, the viral antigen and/or peptide encoded in the coding region CR can comprise, but is not limited to, E6 and/or E7 in HPV, g3 in VZV, VP1 and/or VP2 domains in CVB3, antigens and/or peptides derived from Moneypox virus (MPV) and hemagglutinin (HA) in influenza viruses. The peptide for treating diseases can comprise, but is not limited to, LRS-UNE-L domain for muscular diseases, Fabry AGA (alpha-galactosidase A) for treating rare incurable diseases, PKU PAH (phenylketonuria phenylalanine hydroxylase), PCD10 (Protocadherin10) for suppressing cancers, A20, IK, tIK (truncated IK), PINK1 (PTEN induced kinase 1) and Viemtin for treating autoimmune diseases.

For example, the coding region CR can be derived from a part or all of the genome of human papilloma virus (HPV). The HPV is a virus having circular double helix with 8 kb nucleotides and its genome consist of E1, E2, E4, E6, E7, and L1 and L2. The HPV infects the right epithelial cells of humans as well as mammal, causing various malignant tumors (e.g., cervical cancer, vulvar cancer, vaginal cancer, penile cancer, anal cancer, oral cancer, and oropharyngeal cancer), genital warts, and laryngeal papilloma. Particularly, it has been confirmed that most of cervical cancers are caused by HPV.

The genotype of HPVs is determined by differences in the open reading frame (ORF) of the E6, E7 and L1 genes among the HPV genome sequences. To date, HPV types with more than 170 different genotypes have been discovered. Among them, more than 40 types of HPV are known to be transmitted through sexual contact.

Among the various genotypes of HPV, they are divided into high-risk and low-risk types depending on the risk of causing cervical cancer. In particular, HPV types 16, 18, 31, 33, 35, 39, 45, 51, 52, 56, 58, 59, and 68 belong to the high-risk group and HPV types, and types 6, 11, 26, 34, 40, 42, 44, 47, 53, 66 and 69 belong to the low-risk group are closely related to the progression to cervical cancer. In 2018, it was reported that approximately 569,000 new cervical cancer cases occurred worldwide and approximately 311,000 deaths occurred.

In one embodiment, the coding region CR can be derived from the HPVs or can be tumor suppressive and/or attenuated by modifying a part of nucleotides in the HPV genome. The coding region CR can be derived from a high-risk genome of HPV, such as HPV type 16 and/or HPV type 18, or can consist of a polynucleotide containing a tumor suppressive and/or attenuated portion of the genome of those HPVs.

In one embodiment, the coding region CR can comprise a polynucleotide such as E6 and/or E7 regions of the HPV genome, which acts as oncogenes that promotes tumor growth and malignant transformation, a fragment of those regions, or tumor suppressive or attenuated variants of those regions.

As an example, the polynucleotide encoding the HPV-derived peptide, the fragment thereof, and/or the tumor suppressive or attenuated peptide thereof that can be inserted into the coding region can comprise, but is not limited to, at least one nucleotide sequence of SEQ ID NO: 24, SEQ ID NO: 25, SEQ ID NO: 26, SEQ ID NO: 27, SEQ ID NO: 28, SEQ ID NO: 28, SED ID NO: 29, SEQ ID NO: 30 and SED ID NO: 31.

For example, the polynucleotide of SEQ ID NO: 24 is derived from the polynucleotide encoding the E7 peptide in HPV type 18. The natural polynucleotide encoding the peptide of the E7 region in the HPV type 18 comprise nucleotides encoding cysteine and glutamic acid in the 76-78^{th} nucleotides and 82-84^{th} nucleotides. Each of the 76-78^{th} nucleotides and the 82-84th nucleotides in the polynucleotide of SEQ ID NO: 24 is modified to codons encoding glycine (GGC, GGA) for suppressing tumorigenesis of the E7 region in the HPV type 18.

The polynucleotide of SEQ ID NO: 25 is derived from the polynucleotide encoding the E7 peptide in HPV type 16. The natural polynucleotide encoding the peptide of the E7 region in the HPV type 16 comprise nucleotides encoding cysteine and glutamic acid in the 66-68th nucleotides and 72-74th nucleotides. Each of the 66-68^{th} nucleotides and the 72-74^{th} nucleotides in the polynucleotide of SEQ ID NO: 24 is modified to codons encoding glycine (GGC, GGA) for suppressing tumorigenesis of the E7 region in the HPV type 16.

Each of the polynucleotides of SEQ ID NO: 26, SEQ ID NO: 27 and SED ID NO: 28 is derived from the polynucleotide encoding the E6 peptide in HPV type 16. The natural polynucleotide encoding the peptide of the E6 region in the HPV type 16 comprise nucleotides encoding cysteine in the final three nucleotides (183-185th nucleotides). The 183-185th nucleotides in the polynucleotide of SEQ ID NO: 26 are modified to codons encoding glycine (GGC) for suppressing tumorigenesis of the E6 region in the HPV type 16.

The natural polynucleotide encoding E6 peptide in the HPV type 16 includes nucleotides encoding CPEEK amino acids between the polynucleotide of SEQ ID NO: 27 and the polynucleotide of SEQ ID NO: 28. The polynucleotides of SEQ ID NO: 27 and SEQ ID NO: 28 without the intervening nucleotides can be inserted into the coding region CR for suppressing tumorigenesis of the inserted polynucleotides.

Each of the polynucleotides of SEQ ID NO: 29, SEQ ID NO: 30 and SED ID NO: 31 is derived from the polynucleotide encoding the E6 peptide in HPV type 18. The natural polynucleotide encoding the peptide of the E6 region in the HPV type 18 comprise nucleotides encoding cysteine in the final three nucleotides (190-192^{th} nucleotides). The 190-192^{th} nucleotides in the polynucleotide of SEQ ID NO: 29 are modified to codons encoding glycine (GGC) for suppressing tumorigenesis of the E6 region in the HPV type 18.

The natural polynucleotide encoding E6 peptide in the HPV type 18 includes nucleotides encoding NPAES amino acids between the polynucleotide of SEQ ID NO: 30 and the polynucleotide of SEQ ID NO: 31. The polynucleotides of SEQ ID NO: 30 and SEQ ID NO: 31 without the intervening nucleotides can be inserted into the coding region CR for suppressing tumorigenesis of the inserted polynucleotides.

In one embodiment, when the coding region CR polynucleotide encoding the multiple HPV-derived peptides, a fragment thereof, or tumor-suppressive and/or attenuation (inactivation) peptides thereof, a linker element can be inserted among those polynucleotides. For example, GSGSG liner (e.g., SEQ ID NO: 32) can be inserted among the polynucleotides encoding the HPV-derived peptides, a fragment thereof, or tumor suppressive or attenuation peptide thereof.

There is no limitation in the length of the ORFs in the coding region CR, and the expression efficiency depending on the ORF length is not considered in developing a nucleic acid molecule, a recombinant expression vector, and nucleic acid vaccine for treatment or prevention using the molecule. Codon usage is not considered in developing human vaccines or gene therapies because codon usage basis in human has not influence on common peptides/proteins expression significantly, while codon usage may have an influence on the expression of proteins/peptides in various species. But, it can be necessary that start codon have Kozak sequence and nucleotides adjacent to termination codon may be optimized. If necessary, the third codon among GOI or its transcript mRNA codon to be expressed may be changed “G/C” without changing amino acid so that mRNA may have improved stability.

The nucleic acid NA1 can comprise one or more expression control elements ECE operably linked to the coding region CR. For example, the expression control element ECE can comprise a transcription control element TCCE located upstream of the coding region TCCE and a translation control element TLCE located adjacently to the coding region CR.

The translation control element TLCE can comprise nucleotide element with translation initiation activity operably linked to the coding region inserted as the open reading frame ORF. For example, the translation control element TLCE can comprise an upstream translation control element U-TLCE located upstream of the coding region CR and/or a downstream translation control element D-TLCE located downstream of the coding region CR. In other words, the coding region CR can be located between the upstream translation control element U-TLCE and the downstream translation control element D-TLCE. The upstream translation control element U-TLCE and/or the downstream translation control element D-TLCE can have an important role in improving the translation efficiency of the one or more ORFs or one or more transcripts in the coding region CR and maintaining stably mRNA of the transcript in the cell without decoying.

In one embodiment, the upstream translation control element U-TLCE can be all of part of 5′-untranslated region (5′-UTR) with a cap dependent translation initiation activity. Most eukaryotic mRNAs has 7-methyl guanosine (cap, m7G) at its 5′ terminus. Translation initiation complex recognizes the cap at 5′ terminus and proceed to AUG of initiation codon to initiate protein synthesis. The cap structure at 5′ terminus initiates protein synthesis and prevent the mRNA from being destroyed by nuclease action. Alternatively, the upstream translation control element U-TLCE can be all or part of 5-UTR with IRES (internal ribosomal entry site) activity.

In case of in vitro transcription (IVT), pDNA (plasmid DNA) is linearized by treatment restriction endonucleases, and then m7G(5′)-ppp(5′)G (this can be regular capped analog) prepared is attached to mRNA prepared using an appropriate RNA polymerase to fabricate capped mRNA. Optionally, there is a method of performing in vitro transcription without a cap analog, performing a cap reaction using a commercially available vaccinia virus capping enzyme, and using an ‘anti-reverse’ cap analog (ARCA) to prevent the reverse reaction of the cap. When ARCA is introduced, only 3′-O-methylation of methylated guanosine can bind to the nucleotide of unmethylated guanosine.

As an example, the modified 5′-Cap structures include Cap1 (methylation of the ribose within the adjacent nucleotide of m7G), Cap2 (methylation of the ribose of the second nucleotide downstream of m7G), and Cap3 (methylation of the ribose of the third nucleotide downstream of m7G), Cap4 (methylation of the ribose of the fourth nucleotide downstream of m7G), ARCA (anti-reverse Cap analogue), modified ARCA (e.g., phosphothioate modified ARCA), inosine, N1-methyl-guanosine, 2′-fluoro-guanosine, 7-diaza-guanosine, 8-oxo-guanosine, 2-amino-guanosine, LNA-guanosine, and 2-azido-guanosine.

For example, each of the upstream translation control element U-TLCE and/or the downstream translation control element D-TLCE may have nucleotide elements or a transcript thereof with cap dependent translation initiation activity induced from animal, for example, mammals, particularly primates, and more particularly humans. The upstream translation control element U-TLCE is a region to which the translation initiation complex binds during the translation of peptides and/or proteins expressed from the coding region (CR), and induces cis-acting nucleotide elements inducing the translation of the coding region CR.

In one embodiment, the downstream translation control element D-TLCE can be located to the upstream translation control element U-TLCE. As an example, when the upstream translation control element U-TLCE is all or part of the nucleotide elements with the cap dependent translation initiation activity or IRES, the nucleic acid molecule NA1 can comprise the downstream translation control element D-TLCE located at the downstream of the coding region CR. The nucleic acid molecule NA1 including both the upstream translation control element U-TLCE and the downstream translation control element D-TLCE can further improve the expression efficiency of the ORF in the coding region CR. In other words, the upstream translation control element U-TLCE and/or the downstream translation control element D-TLCE can play an important role in improving the expression efficiency of the ORF of gene of interest GOI in the coding region CR or the transcript thereof, in maintaining stably the mRNA of the transcript in the cell without destroying.

For example, the cap dependent upstream translation control element UTLCE can comprise, but is not limited to, nucleotide elements selected from the group consisting of SEQ ID NO: 1 (derived from human troponin T1 of slow skeletal type, TNNT1), SEQ ID NO: 2 (derived from human albumin, ALB), SEQ ID NO: 3 (derived from human ferritin light chain, FTL), SEQ ID NO: 4 (derived from human C—C motive chemokine ligand 19, CCL19), SEQ ID NO: 5 (derived from human associated migratory cell protein, AAMP), SEQ ID NO: 6 (derived from human ribosomal protein 27, RPS27) and SEQ ID NO: 7 (derived from human defensing alpha 5, DEFA5), or a transcript thereof.

Alternatively, the upstream translation control element U-TLCE can be all or part of 5′-UTR with an Internal Ribosomal Entry Site (IRES) activity. For example, the upstream translation control element U-TLCE can be all or part of 5′-UTR with a viral IRES activity. The upstream translation control element U-TLCE is a region to which a translational initiation complex binds in the course of transition process of the peptide and/or protein expressed from the coding region CR, and the IRES can be a cis-acting nucleotide element inducing translation of the gene of interest GOI by forming secondary and tertiary structures.

IRES has a unique secondary structure or a tertiary structure and can be divided into Group 1, Group 2, Group 3 and Group 4 based on the presence of specific translation factors for the translation and position of the translation initiation codon. The upstream translation control element U-TLCE with the viral IRES activity can comprise any IRES type among the Group 1 to Group 4.

For example, the upstream translation control element U-TLCE with the viral IRES activity can be derived from, but is not limited to, Picornaviridae, Togaviridae, Dicistroviridae, Flaviridae, Retroviridae and/or Herpesviridae.

For example the upstream translation control element U-TLCE with the viral IRES activity of Picornaviridae can be derived from, but is not limited to, viruses in Enterovirus genus, viruses in Cardiovirus genus, viruses in Apthovirus genus, viruses in Hepatovirus genus and/or viruses in Teschovirus genus. As an example, the upstream translation control element U-TLCE with the viral IRES activity can be derived from, but is not limited to, viruses classified as Enterovirus type A to Enterovirus type J and/or viruses classified as Rhitovirus type A to Rhinovirus type C.

In unlimited embodiment, the upstream translation control element U-TLCE with the viral IRES activity of Picornaviridae can comprise, but is not limited to, Poliovirus (PV), Rhinovirus (RV), Coxsachievirus), for example, CoxsachievirusB3 (CVB3), Enterovirus 71 (EV71) of Enterovirus genus; Encephalomyocarditis virus (EMCV), Theiler murine encephalomyocarditis virus (THEV) of Cardiovirus genus; Foot-and-mouth disease virus (FDMV) of Apthovirus genus; Hepatitis A virus (HAV) of Hepatovirus genus; Porcine teschovirus (PTV, for example, PTV-1) of Teschovirus genus; Solenopsis invicta virus (SINV-1) of Aparavirus genus; and combinations thereof.

In another embodiment, the upstream translation control element U-TLCE with the viral IRES activity of Togaviridae can be derived from, but is not limited to, Sindbis virus (SV) of Alphavirus genus. In another embodiment, the upstream translation control element U-TLCE with the viral IRES activity of Dicistroviridae, for example, Cripavirus genus, can be derived from, but is not limited to, Plautia stali intestine virus (PSIV), Cricket paralysis virus (CPV), Triatomavirus and/or Rhopalosiphum padi virus (PXRD).

In another embodiment, the upstream translation control element U-TLCE with the viral IRES activity of Flaviridae can be derived from, but is not limited to, Hepatitis C virus (HCV) of Hepacivirus genus; Japanese Encephalitis virus (JEV) of Flavivirus genus; Classical swine fever virus (CSFV) of Pestivirus genus; and/or Bovine viral diarrheas virus (BVDV). In another embodiment, the upstream translation control element U-TLCE with the viral IRES activity of Retroviridae can be derived from, but is not limited to, Friend murine leukemia virus (FMLV), Moloney murine leukemia virus (HMLV) of Gammaretrovirus genus; and/or Rous sarcoma virus (RSV) of Alpharetrovirus genus. In another embodiment, the upstream translation control element U-TLCE with the viral IRES activity of Herpesviridae can be derived from, but is not limited to, Marek's disease virus (MDV) of Mardivirus genus.

The downstream translation control element D-TLCE can be located at the downstream of the coding region CR correspondingly to the upstream translation control element U-TLCE. The gene of interest can be expressed stably owing to the downstream translation control element D-TLCE.

In one embodiment, the downstream translation control element D-TLCE can be 3′-UTR corresponding to the cap-dependent upstream translation control element U-TLCE. As an example, the downstream translation control element D-TLCE can comprise, but is not limited to, nucleotide elements selected from the group consisting of SEQ ID NO: 8 (derived from TNNT1), SEQ ID NO: 9 (derived from ALB), SEQ ID NO: 10 (derived from FTL), SEQ ID NO: 11 (derived from CCL19), SEQ ID NO: 12 (derived from AAMP), SEQ ID NO: 13 (derived from RPS27) and SED ID NO: 15 (derived from DEFA6), or a transcript thereof.

In another embodiment, the downstream translation control element D-TLCE can be 3′-UTR corresponding to the upstream translation control element U-TLCE with the IRES activity. As an example, the downstream control element D-TLCE can comprise, but is not limited to, any 3′-UTR corresponding to the upstream translation control element U-TLCE with the viral IRES activity.

Alternatively or additionally, the nucleic acid molecule NA1 can further comprise any nucleotide element that can increase the expression efficiency of the coding region CR in the form of the ORFs. As an example, the nucleic acid molecule NA1 can comprise the transcription control element TCCE located adjacently to the upstream translation control element U-TLCE and promoting the transcription of the coding region CR. As an example, the transcription control element TCCE can be located at the upstream of the upstream translation control element U-TLCE. The transcription control element TCCE is not limited to a particular nucleotide.

In one embodiment, the transcription control element TCCE can comprise one or more promoters that promote the transcription of the ORFs in the coding region CR. The transcription control element TCCE can be acted within animal cells, for example, mammalian cells, and can control or regulate the transcription of the genes or a fragment thereof encoded in the coding region CR.

As an example, the transcription control element TCCE can comprise one or more promoters derived from mammalian viruses, ono or more promoters derived from genomes of mammalian cells and/or one or more promoters derived from bacteriophages. For example, the transcription control element TCCE can comprise, but is not limited to, a CMV (cytomegalo virus) promoter, an adenovirus late promoter, a vaccinia virus 7.5K promoter, a SV40 promoter, an HSV tk promoter, a T7 bacteriophage promoter, a T3 bacteriophage promoter, an SM6 promoter, an RSV promoter, an EF1 alpha promoter, a metallothionein promoter, a beta-actin promoter, a promoter of human IL-2, a promoter of human IFN, a promoter of human IL-4, a promoter of human Lymphotoxin, a promoter of human GM-CSF, cancer cell specific promoters (e.g., TERT promoter, PSA promoter, PSMA promoter, CES promoter, E2F promoter and AFP promoter) and tissue specific promoters (e.g., albumin promoter).

In one embodiment, any transcription control element TCCE that can transcribe linearized DNA template into mRNAs, for example, T7 bacteriophage promoter, T3 bacteriophage promoter, SP6 bacteriophage promoter and the like, can be located adjacently to the upstream translation control element U-TLCE, for example, downstream of the upstream translation control element U-TLCE.

In addition, the nucleic acid molecule NA1 other nucleotide elements that can induce the expression of the ORF having one or genes of a transcript thereof in the coding region CR. In one embodiment, Kozak element can be inserted between the upstream translation control element U-TLCE and the start codon in the coding region CR.

Also, the nucleic acid molecule NA1 can comprise one or more cloning sites, for example, Multiple Cloning Site (MCS) to insert the coding region CR within the nucleic acid molecule NA1. One or more cloning sites can comprise one or more restriction endonuclease recognition site and/or cleavage sites. The restriction endonuclease can comprise any natural restriction endonucleases found in bacteria or archaea, as well as artificially fabricated restricted endonucleases (e.g., zinc finger nucleases, restriction endonucleases based on DAN binding sites of TAL effector, or PNA-based PNAzymes).

For example, the natural restriction endonucleases can be classified to 1) Type I restriction endonuclease (cleaving sites spaced apart from the recognition sites and requiring ATP, S-adenosyl-L-methionine and magnesium ion); 2) Type II restriction endonuclease (cleaving sites within the recognition sites or spaced apart from the recognition sites and requiring magnesium ion); 3) Type III restriction endonuclease (cleaving sites spaced apart from the recognition sties, requiring ATP but is not requiring ATP hydrolysis); 4) Type IV restriction endonuclease (requiring modified sites such as methylation, hydroxyl-methylation or glucosyl-hydroxyl-methylation); 5) Type V restriction endonuclease such as cas9-gRNA complex of CRISPRs).

For example, the Multiple Cloning Site (MCS) can comprise, but is not limited to, any sites recognized by or cleaved by the restriction endonuclease selected from AngI, AatI, AbaI, BamHI, BbvI, BcgI, BplI, BsmAI, Alw26I, BsrI, ClaI, Earl, Eco57I, EcoRI, EcoRII, EcoRV, FokI, HaeIII, HindIII, HpaIII, HphI, KpnI, MboI, MluI, NaeI, NdeII, NgoMIV, NlaIII, NotI, PacI, PstI, SacI, SacII, SalI, SfaNI, SmaI, TaqI, XbaI, XhoI, PvuI, and combination thereof.

Alternatively or additionally, the nucleic acid molecule NA1 can further comprise a polyadenylation signal sequence and/or a polyadenosine sequence PA located downstream of the coding region CR, for example, downstream of the downstream translation control element D-TLCE. The polyadenylation signal sequence and/or the polyadenosine sequence PA can stabilize the transcribed nucleic acid molecule NA and can further improved the expression efficiency of ORFs having genetic sequences of a transcript thereof in the coding region CR. In one embodiment, the polyadenylation signal sequence and/or the polyadenosine sequence PA can be located downstream of the coding region CR, for example, between the downstream translation control element D-TLCE and the stem-loop element SL1. In another embodiment, the polyadenylation signal sequence and/or the polyadenosine sequence PA can be located downstream of the stem-loop element SL1.

For example, when the nucleic acid molecule NA1 includes the transcription elements of RNA type, the polyadenosine sequence PA can be nucleotides consisting of about 25 to about 400, for example, about 30 to about 400, about 50 to about 250 or about 60 to about 250 adenosines.

In another embodiment, when the nucleic acid molecule NA1 has a DNA type, the polyadenylation signal sequence PA can be located downstream of the coding region CR. As an example, the polyadenylation signal sequence PA can be, but is not limited to, derived from SV40, human growth factor (hGH), bovine growth factor (BGH) and/or rabbit beta-globin (rbGlob).

In one embodiment, the nucleic acid molecule NA1 can further nucleotide element encoding a signal sequence at an upstream of the coding region CR. The signal sequence can be a fragment directing secretion of biologically active molecule therapeutics and/or peptides, and can be cleaved after translation in a host cell. As an example, the signal sequence can be a polynucleotide encoding amino acids initiating translocation of a peptides passing through an ER (endoplasmic reticulum) membrane.

The signal sequences are known to in the art, and can usually comprise about 16 to about 30 amino acid residues, but can amino acid residues more than 30 and/or less than 16. The common signal peptide can comprise three regions of an N-terminal region, a central hydrophobic region and a more polar C-terminal region. The central hydrophobic region contains 4 to 12 hydrophobic residues that anchor the signal sequences through the membrane lipid bilayer while the immature polypeptide moves. After initiation, the signal peptide is cleaved within the lumen of the ER by cellular enzyme commonly known as signal peptidases. As an example, the signal sequence can comprise, but is not limited to, polynucleotide (e.g., SEQ ID NO: 23) encoding human albumin signal peptide.

The nucleic acid molecule NA1 can have a DNA type of an RNA type. In one embodiment, the nucleic acid molecule NA1 can have an RAN type. When the nucleic acid molecule having the RNA type can have a superior property to the nucleic acid molecule having the DNA type.

The nucleic acid molecule in the form of RNA type does not need to enter the nucleus of the cell for transcription into RNA unlike the nucleic acid molecule in the form DNA type. The nucleic acid molecule in the form of RNA type has no possibility of inserting into the host chromosome within the nucleus, antibiotic resistance genes, which are selection markers used for selective production in host cells, are unnecessary for the fabrication of RNA nucleic acid molecule, and RNA does not induce persistent genetic transformation because RNA has a short half-life compared to DNA.

RNA type nucleic acid molecule can induce a desired in vivo immune response even when used in relatively small amounts compared to the DNA type nucleic acid molecule. In addition, the RNA type nucleic acid molecule can be safely produced in small-scale GMA (good manufacturing practices) production facilities without the risk of biological contamination because all producing processes can be artificially controlled in producing nucleic acid molecules in the form of RNA type. Three is no need to directly deal with infections agents in producing RNA type nucleic acid molecule, instead, only the nucleic acid sequence of the part related to the induction of neutralizing antibodies (neutralizing epitopes) of the infection agents to be expressed is artificially synthesized and considerably amount of RNA can be produced through in vitro transcription (IVT). Recently, as reagents related to IVT reactions, particularly, DNA-dependent RNA polymerase, have been improved, it is possible to rapidly produce large amounts of RNA within 1 to 2 weeks using small amount of DNA template.

The nucleic acid molecule in the form of RNA type can induce a stronger immune response than naked DNA type nucleic acid molecules, and the RNA type nucleic acid molecule itself can produce a complex antigens within the cell, and the complex antigens can approach a major histocompatibility complex (MHC) class II of the antigen-presenting cells and can function as an ideal adjuvant. In addition, multiple antigens induce immune responses can be produced simultaneously, mixed, and then immunized, and there are no particular restrictions on the gene length of the antigen to be expressed, which can increase the applicability and simplicity of the RNA type nucleic acid molecule productions.

If one wishes to utilize a nucleic acid molecule of the RNA type, an appropriate transcriptional regulatory element (TCCE) enabling IVT can be placed upstream of the upstream translational control element (U-TLCE). Since the nucleic acid molecules of RNA type can be synthesized through the IVT process, there is no need to directly deal with live viruses or pathogenic microorganisms used in the production of general live or killed vaccines, and there is no need to culture of host cells such as yeast, E. coli or insect cells, which must be used to produce recombinant proteins/peptides.

When transcribing a DNA-type nucleic acid molecule inserted into a vector into an mRNA form through the IVT process, the linearized DNA whose ends are cleaved by restriction endonucleases is used to as a template to synthesize the RNA-type nucleic acid molecule by RNA polymerase in vitro. The transcription control element TCCE such as a promoter element derived from, for example, a bacteriophage can be located upstream of the translation control element TLCE.

FIG. 2 is a schematic diagram illustrating component of a nucleic acid molecule or a polynucleotide that can express a gene of interest efficiently in accordance with another embodiment of the present disclosure. A nucleic acid molecule NA2 illustrated in FIG. 2 includes a downstream translation control element D-TLCE with multiple elements located downstream of the coding region CR comparing the nucleic acid molecule NA1 illustrated in FIG. 1. The downstream translation control element D-TLCE comprises a first downstream translation control element D-TLCE1 and a second downstream translation control element D-TLCE2 located sequentially downstream of the coding region CR.

In one embodiment, each of the first downstream translation control element D-TLCE1 and the second downstream translation control element can independently comprise 3′-UTR corresponding to the cap-dependent upstream translation control element U-TLCE. The first downstream translation control element D-TLCE1 and the second downstream translation control element D-TLCE2 can be identical to or different from each other. As an example, each of the first downstream translation control element D-TLCE1 and the second downstream translation control element D-TLCE2 can comprise independently, but is not limited to, nucleotide element selected from the group consisting of SEQ ID NO: 8 (derived from TNNT1), SEQ ID NO: 9 (derived from ALB), SEQ ID NO: 10 (derived from FTL1), SEQ ID NO: 11 (derived from CCL19), SED ID NO: 12 (derived from AAMP), SEQ ID NO: 13 (derived from RPS27) and SED ID NO: 14 (derived form DEFA5), or a transcript thereof.

In another embodiment, each of the first downstream translation control element D-TLCE1 and the second downstream translation control element D-TLCE2 can independently comprise 3′-UTR corresponding to the upstream translation control element U-TLCE with the IRES activity. For example, each of the first downstream translation control element D-TLCE1 and the second downstream translation control element D-TLCE2 can comprise, but is not limited to, a virus-derived 3′-UTR corresponding to the upstream translation control element U-TLCE with the viral IRES activity.

Two independent first and second downstream translation control elements D-TLCE1 and D-TLCE2 are illustrated in FIG. 2. In another embodiment, the downstream translation control element D-TLCE can further one or more downstream translation control elements such as a third downstream translation control element located downstream of the second downstream translation control element D-TLCE2.

For example, the third downstream translation control element can comprise 3′-UTR corresponding to the cap-dependent upstream translation control element U-TLCE or 3′-UTR corresponding to the upstream translation control element U-TLCE with the IRES activity (e.g., the viral IRES activity). For example, the third downstream translation control element can comprise, but is not limited to, the nucleotide element selected from the group consisting of SEQ ID NO: 8 to SEQ ID NO: 14, or a transcript thereof.

The stem-loop element SL1 is located adjacently 3′ terminus of the nucleic acid molecule NA2. Alternatively, the polyadenylation signal sequence and/or the polyadenosine sequence PA can be located downstream of the stem-loop element SL1.

FIG. 3 is a schematic diagram illustrating component of a nucleic acid molecule or a polynucleotide that can express a gene of interest efficiently in accordance with another embodiment of the present disclosure. Each of a first linker element LK1 and a second linker element LK2 is inserted between the first stem element ST1 and the loop element L, and the loop element and the second stem element ST2, respectively, within a stem-loop element SL2 constituting a nucleic acid molecule NA3 in FIG. 3 comparing the nucleic acid molecule NA1 illustrating in FIG. 1. More particularly, comparing the stem-loop element SL1 in FIG. 1, the stem-loop element ST2 further includes the first linker element LK1 between the first stem element ST1 and the loop element L and the second linker element LK2 between the loop element L and the second stem element ST2.

Each of the first linker element LK1 and the second linker element LK2 can comprise any nucleotides of about 5 to about 50, for example, about 5 to about 30, about 5 to about 20, or about 5 to about 15. For example, about 90% or more, for example, about 80% or more, about 70% or more, about 60% or more, about 50% or more, about 40% or more, about 30% or more, about 20% or more, or about 10% or more of the nucleotides in each of the first linker element LK1 and the second linker element LK2 cannot be complementary to each other. The expression efficiency of the gene of interest encoded in the coding region CR can be further improved by inserting the linker elements LK1 and LK2 among the stem elements ST1 and ST2 and the loop element L.

FIG. 4 is a schematic diagram illustrating component of a nucleic acid molecule or a polynucleotide that can express a gene of interest efficiently in accordance with another embodiment of the present disclosure. A nucleic acid molecule NA4 illustrated in FIG. 4 includes a downstream translation control element D-TLCE with multiple elements located downstream of the coding region CR comparing the nucleic acid molecule NA3 illustrated in FIG. 3. The downstream translation control element D-TLCE in FIG. 4 comprises a first downstream translation control element D-TLCE1 and a second downstream translation control element D-TLCE2 located sequentially downstream of the coding region CR. In another embodiment, the downstream translation control element D-TLCE can further one or more downstream translation control elements such as a third downstream translation control element located downstream of the second downstream translation control element D-TLCE2.

The transcription control element TCCE, the upstream translation control element U-TLCE, the coding region, the first to third downstream translation control element D-TLCE and the polyadenylation signal sequence and/or the polyadenosine sequence PA and the stem-loop element illustrated in FIGS. 3 and 4 can be identical to the corresponding configurations with referring to FIGS. 1 and 2.

### Expression Construct (Expression Vector) and Nucleic Acid Transfection or Injection

The nucleic acid molecules (hereinafter, “NA”) itself can be transfected into a host cells, or the nucleic acid molecule NA1 etc. can be inserted into a vector and then an expression construct or an expression vector of a gene carrier can be transfected into the host cells.

The vector that can be used as the gene carrier can be fabricated with various formats, and can comprise, but is not limited to, viral vectors, DNA or RNA expression vectors, plasmid, cosmid or phage vectors, DNA or RNA expression vectors linked to CCA (cationic condensing agents), for example, DNA or RNA expression vectors packaged with liposome or niosome including plasmids, and/or particular eukaryotic cells such as producer cells.

In one embodiment, the nucleic acid molecule NA can have compositions for transfected into mammalian cells to be expressed. Such compositions can be useful for the purposes of treatment and/or prevention of diseases. There are various techniques in expressing the nucleic acid molecule NA within the host cells, and any appropriate techniques can be used. For example, the nucleic acid molecule NA can be applied into any expression constructs or gene delivery system.

A category of a vector is a ‘plasmid’ which refers to a circular, double-stranded DNA loop into which additional nucleic acid molecule may be ligated. Another category of vector is a phage vector. Still another category of vector is viral vectors into which additional nucleic acid molecule may be ligated into the viral genome. Specific vectors can replicate autonomously into the host cells having the transfected the vectors (e.g. viral vectors and episome mammalian vectors having bacterial replication origins). Other vectors (e.g. non-episome mammalian vectors) may be integrated into the genome of a host cell as they transfect the host cell, and thereby, being replicated together with the genome of the host cell. Besides, specific vectors may direct the expression of genes operably linked to the vectors. Such vectors are referred herein as a “recombinant expression vector (or, shortly, “recombinant vector”). Generally, the expression vectors, which may be useful for recombinant DNA technologies, exist as a shape of plasmid.

In another embodiment, the nucleic acid molecule NA can be transfected into the host cells using viral gene delivery systems. The viral vector into which the nucleic acid molecule can be inserted can comprise, but is not limited to, vectors derived from adenovirus, adeno-associated viruses (AAVs), retrovirus, vaccinia or other pox viruses (e.g., avian virus), lentiviruses and/or herpes simplex viruses. For example, the viral vector can comprise, but is not limited to, retrovirus vectors derived from lentiviruses such as human immunodeficiency virus (HIV) and simian immunodeficiency virus (SIV); and retroviruses such as murine retroviruses, gibbon ape leukemia virus, AAVs and adenoviruses. In addition, viral vectors derived from murine leukemia virus (MuLV) and ecotropic retroviruses can be used. The vector system can be constructed by various techniques known in the art.

In one embodiment, the nucleic acid molecule NA is inserted appropriate vectors and then modified RNA transcript via in vitro transcription (IVT).

It has already been well-known that techniques of inserting nucleic acid molecules, for example DNA, into such vectors. It is possible to insert additionally targeting moieties such as selection marker genes for making easy certification or selection for the transfected cells and/or genes encoding ligands acting as a receptor to a particular target cell in the retrovirus vector. Targeting may be performed by known processes using specific antigens.

It is possible to use plural vectors that are commercially available and known to in the art for the purposes of the present disclosure. Selecting appropriate vectors will be mainly dependent upon the sizes of the nucleic acid molecules to be inserted into the vectors and specific host cells transfected with the vectors. Each vector contains various components, depending upon its functions (amplification and/or expression of foreign polynucleotides) and compatibilities to the specific host cells having thereof. Vector components generally comprises, but are not limited to, replication origins (especially if the vector is inserted into prokaryotes), selection marker genes, promoters, ribosome binding sites (RBS), signal sequences, foreign nucleic acids insert, and a transcription termination sequence.

For example, the expression vector of the present disclosure can comprise other expression control sequences, which may have an effect on the expression of the genes of ORF type encoded in the coding region CR or a transcript thereof, for example, an initiation codon, a termination codon, one or more enhancers, and/or signal sequences for membrane-targeting or secretions, and the likes. Enhancer sequences are nucleic acid sequences which are located at various sites with regard to transcription control sequence, e.g. promoter and increase transcription activity compared to a transcription activity by the promoter without the enhancer sequences.

Signal sequences comprise, but are not limited to, PhoA signal sequence, OmpA signal sequence, and the likes in case the host cell is bacteria in Escherichia spp., α-amylase signal sequence, subtilisin sequence and the likes in case the host cell is bacteria in Bacillus spp., MF-α signal sequence, SUC2 signal sequence and the likes in case the hose cell is yeast, and insulin signal sequence, α-interferon signal sequence, antibody molecule signal sequence and the likes in case the host cell is mammals.

The vector can be constructed as a vector for cloning or a vector for expression. In addition, the vector can be constructed for the prokaryotic host cells or the eukaryotic host cells. For example, the vector of the present disclosure can be constructed by manipulating the usually used vectors in the art, plasmid (e.g., pSC101, ColE1, pBR322, pUC8/9, phC79, pUC19, pET and the likes), phage (e.g., λgt4 λB, λ-Charon, λΔz1, λGEM™-11, M13 and the likes), virus (e.g., SV40 and the likes).

Constitutively or inducible promoters can be used as the transcription control sequence TCES in the present disclosure. Plural promoters that recognized by various possible host cells have been widely known in the art. Selected promoters may be operably linked to the nucleic acid molecule having the coding region CR comprising ORF of appropriate GOI encoding one or more peptides or proteins by removing the promoters from supplier nucleic acid molecule through restriction endonuclease digestions and then inserting the isolated promoter sequences into the selection vectors. It is possible to direct amplification and/or expression of the target genes using both natural promoter sequences and a plurality of foreign promoters. But, foreign promoters are generally more preferable to the natural targeting polypeptide promoters because the foreign promoters allows much transcription and high yield of the expressed target genes compared to the natural targeting polypeptide promoters.

For example, when the vector of the present disclosure is an expression vector and a host cell is eukaryotes, the vector can comprise, but is not limited to, strong promoters for proceeding with the transcription (e.g., tac promoter, lac promoter, LacUV5 promoter, lpp promoter, pLλ promoter, pRλ promoter, rac5 promoter, amp promoter, recA promoter, SP6 promoter, trp promoter and T7 promoter), at least one of the expression control sequences TLCE and transcription/translation termination sequences. In case of using E. coli as the host cell, promoters and operator sites of E. coli tryptophan biosynthetic pathway can be used as a control site.

Alternatively, when the vector of the present disclosure is an expression vector and uses eukaryotes as the host cell, the vector can comprise, but is not limited to, promoters such as promoters derived from the genome of the mammalian cells (e.g., metallothionein promoter), promoters derived from mammalian viruses (e.g. adenovirus late promoter, vaccinia virus 7.5K promoter, SV40 promoter, cytomegalovirus promoter and tK promoter of HSV) or promoters derived from phages (e.g. T7 promoter, T3 promoter and SM6 promoter), and polyadenylation signal sequences as a transcription termination signal sequence.

In addition, when the recombinant vector of the present disclosure is a replicable repression vector, it may comprise a replication origin, which is a specific nucleic acid sequence for initiating replication. In addition, the recombinant vectors may comprise sequences encoding selectable markers. The selectable markers are intended to screen transfected cells by the vectors and markers giving selectable phenotypes such as drug resistances, nutritional requirements, cytotoxic agent resistances, or expressions of surface proteins may be used. The vectors of the present invention may comprise antibiotics resistant genes which have been conventionally used in the art, for example, ampicillin, gentamicin, carbenicillin, chloramphenicol, streptomycin, kanamycin, geneticin, neomycin, and tetracycline resistant genes as selectable markers. It is possible to screen the transfected cells because only cells expressing the selectable markers can survive in an environment of treating elective agents. Representative example of the selectable markers may comprise an auxotrophic marker, ura4, leu1, his3 and the likes, but the selectable markers can be used in the present invention is not limited to such an example.

Various in vitro amplification techniques that amplify sequences sub-cloned to expression vectors have been known. There are PCR (polymerase chain reaction), LCR (ligase chain reaction), Qβ-replicase amplification and techniques using other RNA polymerases in such techniques

The vector of the present disclosure can be fused with other sequences in order to facilitate the purification of the peptide expressed therefrom. Fused sequences comprises glutathione S-transferase (Pharmacia, USA), maltose binding protein (NEB, USA), FLAG (IBI, USA), 6×His (hexahistidine, Quiagen, USA) and the likes, and 6×His is most preferable. Owing to the additional sequences for purification, the proteins expressed in the host cell are purified with promptness and ease through affinity chromatography assay. If necessary, a sequence encoding Fc fragments may be fused with the vector in order to facilitate extracellular secretion of those peptides.

In one embodiment, the peptides expressed by the vector which comprises the nucleic acid molecule are purified by affinity chromatography. For example, it is possible to purify the peptides or proteins of interest with promptness and with ease by using glutathione as a substrate of glutathione-S-transferase when glutathione-S-transferase is fused with the vector, and by using Ni-NTA His-binding resin column (Novagen, USA) when the vector comprises 6×his.

It is possible to use any host cells known in the art as long as the host cells make the vectors stably and continuously clone and express. For example, host cells may comprise E. coli JM109, E. coli BL21 (DE3), E. coli RR1, E. coli LE392, E. coli B, E. coli×1776, E. coli W3110, Bacillus strains such as Bacillus subtilis, Bacillus thuringiensis, and enterobacteriaceae strains such as Salmonella typhimurium, Serratia marcescens and various Pseudomonas species. Besides, yeast (Saccharromyce cerevisiae), insect cells (e.g., SF9 cell), human cells (e.g., CHO (Chinese hamster ovary) cell, W138, BHK, COS-7, 293, HepG2, 3T3, RIN and MDCK cell lines) may be used as host cells in case of transfecting eukaryotes with the vectors of the present invention.

The vectors of the present disclosure may be used in modifying genetically the host cells in in vivo or ex vivo or in in vitro. With regard to methods of modifying genetically cells, various methods comprising cell transfection or transduction with viral vectors, calcium phosphate precipitation, methods of fusing recipient cells with bacterial protoplast containing DNA, method of treating recipient cells with liposome or microspheres containing DNA, DEAE dextran, receptor mediated endocytosis, electroporation, micro-injection, and gene bombardment are well known in the art.

For example, when the host cells are eukaryotes, the vector may be injected into the host cells by Hanahn method and/or electroporation method. In addition, when the host cells are eukaryotes, the vector may be injected into the host cells by micro-injection method, calcium phosphate precipitate method, electroporation method, liposome-mediated transfection method, DEAE-dextran treatment method, and gene bombardment method. The vector injected into the host cells may be expressed within the cell in which large amount of recombinant peptides or proteins are obtained. For example, when the expression vector includes lac promoter, it is possible to induce gene expression by treating IPTG to the host cells.

### Composition

The present disclosure provides a composition including the nucleic acid molecule NA or the expression construct (gene delivery system) into which the nucleic acid molecule NA is inserted.

In one embodiment, the nucleic acid molecule NA and/or the expression construct into which the nucleic acid molecule NA is inserted can be utilized as a detection composition for detecting or analyzing specific material or molecule in a sample when the coding region CR in the nucleic acid molecule NA encodes a report peptide, a marker or a selection peptide.

In another embodiment, the nucleic acid molecule NA and/or the expression construct into which the nucleic acid molecule NA is inserted can be utilized as a pharmaceutical composition for the treatment and/or prevention of diseases when the coding region CR in the nucleic acid molecule NA encodes one or more peptides or a fragment thereof associated with the prevention and/or treatment of diseases.

For example, the nucleic acid molecule NA and/or the expression construct into which the nucleic acid molecule NA is inserted can be utilized as a pharmaceutical composition for the treatment and/or the prevention of cancer or tumor diseases, infectious diseases, autoimmune diseases, allergy or allergic diseases, genetic disorders, cardiovascular diseases, nervous system diseases, circulatory system diseases, respiratory system diseases, digestive system diseases, skin and subcutaneous tissue diseases, musculoskeletal diseases, connective tissue diseases, immune deficiency, endocrine, nutrimental and metabolic diseases, eye diseases and/or ear diseases.

For example, cancer or tumor diseases can comprise, but is not limited to, colon carcinomas, melanomas, renal carcinomas, lymphomas, acute myeloid leukemia (AML), acute lymphoid leukemia (ALL), chronic myeloid leukemia (C-ML), chronic lymphocytic leukemia (CLL), gastrointestinal tumors, pulmonary carcinomas, gliomas, thyroid tumors, mammary carcinomas, prostate tumors, hepatomas, various virus-induced tumors such as, for example, papilloma virus-induced carcinomas (e.g. cervical carcinoma), adenocarcinomas, herpes virus-induced tumors (e.g. Burkitt's lymphoma, EBV-induced B-cell lymphoma), hepatitis B-induced tumors (hepatocell carcinoma), HTLV-1- and HTLV-2-induced lymphomas, acoustic neuromas/neurinomas, cervical cancer, lung cancer, pharyngeal cancer, anal carcinomas, glioblastomas, lymphomas, rectal carcinomas, astrocytomas, brain tumours, stomach cancer, retinoblastomas, basaliomas, brain metastases, medulloblastomas, vaginal cancer, pancreatic cancer, testicular cancer, melanomas, thyroidal carcinomas, bladder cancer, Hodgkin's syndrome, meningiomas, Schneeberger disease, bronchial carcinomas, hypophysis tumor, Mycosis fungoides, oesophageal cancer, breast cancer, carcinoids, neurinornas, spinaliomas, Burkitt's lymphomas, laryngeal cancer, renal cancer, thymomas, corpus carcinomas, bone cancer, non-Hodgkin's lymphomas, urethral cancer, CUP syndrome, head/neck tumours, oligodendrogliomas, vulval cancer, intestinal cancer, colon carcinomas, oesophageal carcinomas, wart involvement, tumours of the small intestine, cramopharyngeomas, ovarian carcinomas, soft tissue tumours/sarcomas, ovarian cancer, liver cancer, pancreatic carcinomas, cervical carcinomas, endometrial carcinomas, liver metastases, penile cancer, tongue cancer, gall bladder cancer, leukemia, plasmocytomas, uterine cancer, lid tumor, prostate cancer, and the likes.

In another embodiment, the infectious diseases can comprise, but is not limited to, viral infectious diseases such as influenza, malaria, SARS, yellow fever, AIDS, Lyme borreliosis, Leishmaniasis, anthrax, meningitis, Condyloma acuminata, bollow warts, Dengue fever, three-day fever, Ebola virus, cold, early summer meningoencephalitis (FSME), flu, shingles, hepatitis, herpes simplex type I, herpes simplex type II, Herpes zoster, influenza, Japanese encephalitis, Lassa fever, Marburg virus, measles, foot-and-mouth disease, mononucleosis, mumps, Norwalk virus infection, Pfeiffer's glandular fever, smallpox, polio (childhood lameness), pseudo-croup, fifth disease, rabies, waits, West Nile fever, chickenpox, cytomegalic virus (CMV), Middle east respiratory syndrome (MERS), corona virus infectious disease (COVID-19), zika virus infectious disease; and/or bacterial infectious diseases such as miscarriage (prostate inflammation), anthrax, appendicitis, borreliosis, botulism, Camphylobacter, Chlamydia trachomatis (inflammation of the urethra, conjunctivitis), cholera, diphtheria, donavanosis, epiglottitis, typhus fever, gas gangrene, gonorrhoea, rabbit fever, Heliobacter pylori, whooping cough, climatic bubo, osteomyelitis, Legionnaire's disease, leprosy, listeriosis, pneumonia, meningitis, bacterial meningitis, anthrax, otitis media, Mycoplasma hominis, neonatal sepsis (Chorioamnionitis), noma, paratyphus, plague, Reiter's syndrome, Rocky Mountain spotted fever, Salmonella paratyphus, Salmonella typhus, scarlet fever, syphilis, tetanus, tripper, tsutsuganushi disease, tuberculosis, typhus, vaginitis (colpitis), soft chancre, and from infectious diseases caused by parasites, protozoa or fungi, such as amoebiasis, bilharziosis, Chagas disease, athlete's foot, yeast fungus spots, scabies, malaria, onchocercosis (river blindness), or fungal diseases, toxoplasmosis, trichomoniasis, trypanosomiasis (sleeping sickness), visceral Leishrnaniosis, nappy/diaper dermatitis, schistosomiasis, fish poisoning (Ciguatera), candidosis, cutaneous Leishmaniosis, lambliasis (giardiasis), or sleeping sickness, or from infectious diseases caused by Echinococcus, fish tapeworm, fox tapeworm, canine tapeworm, lice, bovine tapeworm, porcine tapeworm, miniature tapeworm.

In another embodiment, the autoimmune diseases can comprise, but is not limited to, systemic syndromes, including SLE, Sjogren's syndrome, Scleroderma, Rheumatoid Arthritis and polymyositis or local syndromes, including endocrinologic (DM Type 1, Hashimoto's thyroiditis, Addison's disease and the likes), dermatologic (pemphigus vulgaris), haematologic (autoimmune haemolytic anaemia), neural (multiple sclerosis) or can involve virtually any circumscribed mass of body tissue.

For example, the autoimmune diseases can comprise, but is not limited to, multiple sclerosis (MS), rheumatoid arthritis, diabetes, type I diabetes (Diabetes mellitus), systemic lupus erythematosus (SLE), chronic polyarthritis, Basedow's disease, autoimmune forms of chronic hepatitis, colitis ulcerosa, type I allergy diseases, type II allergy diseases, type III allergy diseases, type IV allergy diseases, fibromyalgia, hair loss, Bechterew's disease, Crohn's disease, Myasthenia gravis, neurodermitis, Polymyalgia rheumatica, progressive systemic sclerosis (PSS), psoriasis, Reiter's syndrome, rheumatic arthritis, psoriasis, vasculitis, and the like.

The allergy or the allergic diseases can comprise, but is not limited to, allergic asthma, allergic conjunctivitis, allergic rhinitis anaphylaxis, angiodema, atopic dermatitis, skin allergies, contact dermatitis, and the likes.

For example, the nucleic acid molecule NA and/or the expression construct including the nucleic acid molecule NA can be included as an active ingredient in the pharmaceutical composition for the treatment and/or preventing tumors such as cervical cancer, vulvar cancer, vaginal cancer, penile cancer, anal cancer, oral cancer, and oropharyngeal cancer caused by infection with HPV; chickenpox and/or shingles caused by infection with VZV; myocarditis, meningitis, encephalitis, and/or Type 1 diabetes caused by infection with Enteroviruses such as CVB3; smallpox such as monkeypox caused by infection with MPV; flu caused by infection with the influenza virus; muscle diseases associated with LRS-UNE-L (e.g., hypertrophy, muscular atrophy, muscular dystrophy, myasthenia gravis, sarcopenia), rare refractory diseases associated with Fabry AGA (e.g., Fabry disease), inherited metabolic disorders associated with PHU PAH (e.g., phenylketonuria), developmental and epileptic encephalopathies (DEE) associated with PCDH10; Landau-Klefner syndrome; autoimmune diseases related to autism and/or tumors, A20, IK, tIK, Vimentin, etc. (e.g. rheumatoid arthritis); cataracts; Crohn's disease; and the like.

In one embodiment, the pharmaceutical composition such as a vaccine composition can comprise a pharmaceutically effective amount of the nucleic acid molecule NA or a pharmaceutically effective amount of the expression construct (gene delivery system) including the nucleic acid molecule, and a pharmaceutically acceptable carrier. As used herein, the term “pharmaceutically effective amount” or “therapeutically effective amount” means an amount of sufficiently accomplishing efficacy or activation of an active ingredient, the nucleic acid molecule NA.

As an example, the nucleic acid molecule NA itself or the gene carrier can be administered to a subject. In accordance with another aspect, the present disclosure relates to a method for treating or preventing diseases comprising administering a pharmaceutically effectively amount of the nucleic acid molecule NA or the gene carrier, or a use of the nucleic acid molecule NA and/or the gene carrier for treating or preventing diseases.

In addition, the pharmaceutical composition can further comprise a stabilizer such as a cationic polymer, a cationic peptide or a cationic polypeptide for stabilizing the nucleic acid molecule NA or the expression construct; one or more adjuvants for enhancing immune responses; sustained-release formulation; and one or more lipid nano particles (LNPs) for protecting the active ingredient, the nucleic acid molecule NA and improving injection activity of the nucleic acid molecule into a body.

As an example, the pharmaceutical composition can comprise a nucleic acid stabilizer. As an example, the nucleic acid molecule can be stabilized in the pharmaceutical composition such as the vaccine composition using the cationic polymer, the cationic peptide and/or the cationic polypeptide. The cationic (poly) peptide as the stabilizer can include multiple cationic polymers such as poly-lysine or poly-arginine, cationic lipids and lipofectants. For example, the stabilizer can comprise, but is not limited to, histones, nucleolines, protamines, oligofectainines, spermine or spermidine, cationic polysaccharides, in particular chitosan, TDM, MDP, muramyl dipeptide, pluronics, and/or derivatives thereof. Histones and protamines are cationic proteins which naturally compact DNA. Histones which may be used to form a complex with the nucleic acid molecule can include histones H1, H-1.2a, 1-1D and 14, and protamines which may be used to form a complex with the nucleic, acid molecule can include protamine P1 or P2 or cationic partial sequences of protamine.

The pharmaceutical composition can further other compounds or ingredients. For example, other compounds which may be used to form a complex with the nucleic acid molecule can comprise adjuvants. The adjuvant can enhance immunological activities of the nucleic acid molecule NA and/or the gene carrier that is used as the active ingredient in the pharmaceutical composition.

As an example, the adjuvant which can be contained in the pharmaceutical composition can comprise, but is not limited to, stabilizing cationic peptides or polypeptides such as protamine, nucleoline, spermine or spermidine, and cationic polysaccharides, in particular chitosan, TDM, MDP, muramyl dipeptide, pluronics, alum solution, aluminium hydroxide, ADJUMER (polyphosphazene); aluminium phosphate gel; glucans from algae; algammulin; aluminium hydroxide gel (alum); highly protein-adsorbing aluminium hydroxide gel; low viscosity aluminium hydroxide gel; AF or SPT (emulsion of squalane (5%), Tween 80 (0.2%), Pluronic L121 (1.25%), phosphate-buffered saline, pH 7.4); AVRIDINE (propanediamine); BAY R005 ((N-(2-deoxy-2-L-leucylamino-b-D-glucopyranosyl)-N-octadecyldodecanoyl-amide hydroacetate); CALCITRIOL (1α, 25-dihydroxy-vitamin D3); calcium phosphate gel; CAP™ (calcium phosphate nanoparticles); cholera holotoxin, cholera-toxin-A1-protein-A-D-fragment fusion protein, sub-unit B of the cholera toxin; CRL 1005 (block copolymer P1205); cytokine-containing liposomes; DDA (dimethyldioctadecylammonium bromide); DHEA (dehydroepiandrosterone); DMPC (dimyristoylphosphatidylcholine); DMPG (dimyristoylphosphatidylglycerol); DOC/alum complex (deoxycholic acid sodium salt); Freund's complete adjuvant; Freund's incomplete adjuvant; gamma inulin; Gerbu adjuvant (mixture of i) N-acetylglucosaminyl-(P1-4)-N-acetylmuramyl-L-alanyl-D-glutamine (GMDP), ii) dimethyldioctadecylammonium chloride (DDA), iii) zinc-L-proline salt complex (ZnPro-8); GM-CSF); GMDP (N-acetylglucosaminyl-(bl-4)-N-acetylmuramyl-L-alanyl-D-isoglutamine); imiquimod (1-(2-methypropyl)-1H-imidazo[4,5-c]quinoline-4-amine); ImmTher (N-acetylglucosaminyl-N-acetylmuramyl-L-Ala-D-isoGlu-L-Ala-glycerol dipalmitate); DRVs (immunoliposomes prepared from dehydration-rehydration vesicles); interferon-gamma; interleukin-1-beta; interleukin-2; interleukin-7; interleukin-12; ISCOMS (“Immuno-stimulating Complexes”); ISCOPREP 7.0.3; liposomes; LOXORIBINE (7-allyl-8-oxoguanosine (guanine)); LT oral adjuvant (E. coli labile enterotoxin-protoxin); microspheres and microparticles of any composition; MF59™; (squalene-water emulsion); MONTANIDE ISA 51 (purified incomplete Freund's adjuvant); MONTANIDE ISA 720 (metabolisable oil adjuvant); MPL (3-Q-desacyl-4′-monophosphoryl lipid A); MTP-PE and MTP-PE liposomes ((N-acetyl-L-alanyl-D-isoglutaminyl-L-alanine-2-(1,2-dipalmitoyl-sn-glycero-3-(hydroxyphosphoryloxy))ethylamide, monosodium salt); MURAMETIDE (Nac-Mur-L-Ala-D-Gln-OCH3); MURAPALMITINE and D-MURAPALMITINE (Nac-Mur-L-Thr-D-isoGln-sn-glyceroldipalmitoyl); NAGO (neuraminidase-galactose oxidase); nanospheres or nanoparticles of any composition; NISVs (non-ionic surfactant vesicles); PLEURAN (beta-glucan); PLGA, PGA and PLA (homo- and copolymers of lactic acid and glycolic acid; micro-/nano-spheres); PLURONIC L121; PMMA (polymethyl methacrylate); PODDS (proteinoid microspheres); polyethylene carbamate derivatives; poly-rA: poly-rU (polyadenylic acid-polyuridylic acid complex); polysorbate 80 (Tween 80); protein cochleates (Avanti Polar Lipids, Inc., Alabaster, Ala.); STIMULON (QS-21); Quil-A (Quil-A saponin); S-28463 (4-amino-otec-dimethyl-2-ethoxymethyl-1H-imidazo[4,5-c]quinoline-1-ethanol); SAF-1 (“Syntex adjuvant formulation”); Sendai proteoliposomes and Sendai-containing lipid matrices; Span-85 (sorbitan trioleate); Specol (emulsion of Marcol 52, Span 85 and Tween 85); squalene or Robane (2,6,10,15,19,23-hexamethyltetracosan and 2,6,10,15,19,23-hexamethyl-2,6,10,14,18,22-tetracosahexane); stearyltyrosine (octadecyltyrosine hydrochloride); Theramid (N-acetylglucosaminyl-N-acetylmuramyl-L-Ala-D-isoGlu-L-Ala-dipalmitoxypropylamide); Theronyl-MDP (Termurtide or [thr 1]-MDP; N-acetylmuramyl-L-threonyl-D-isoglutamine); Ty particles (Ty-VLPs or virus-like particles); Walter-Reed liposomes (liposomes containing lipid A adsorbed on aluminum hydroxide), and the likes.

For example, the adjuvant which may be contained in the pharmaceutical composition can include alum (inducing Th₂ immune response and enhancing humoral immune responses); oil-in-water emulsion type adjuvant (enhancing antigenic immune responses and inducing Th₁ immune responses in balance) such as MF59, AS03 and AS04 (mixing MPL of TLR-4 agonist with alum, GSK), AddaVax (squalene base, InvivoGen); LPS (lipid polysaccharides) as agonist to pattern recognition receptors (PRRs) such as Toll-like receptors (TLRs), RIG-I-like receptors (RLRs), NOD-like receptors (NLRs); Poly: C; imidazoquinolines (imiquinod or R848); and/or CpG oligonucleotides.

If necessary, the pharmaceutical composition can further comprise the sustained-release formulations. Suitable examples of sustained-release formulations include semipermeable matrices of solid hydrophobic polymers containing nucleic acid molecules NA or gene carriers, which matrices are in the form of molded articles, for example, films or microcapsules. Examples of sustained-release matrices include polyesters, hydrogels (e.g., poly(2-hydroxyethyl-methacrylate), or poly(vinyl alcohol)), polylactide, L-glutamic acid, and gamma-ethyl-copolymers of L-glutamate, non-degradable ethylene-vinyl acetate, degradable lactic acid-glycolic acid copolymers and poly-D-(−)-3-hydroxybutyric acid.

In another embodiment, the pharmaceutical composition can comprise one or more lipid nanoparticles (LNPs) protecting the nucleic acid molecule NA or the expression construct or gene carrier as the active ingredient and improving the injection of active ingredient to a body. In one embodiment, the lipid nanoparticle can comprise multiple lipid molecules physically assembled each other, and can comprise microsphere (mono-layered or multi-layered vesicles such as liposomes), a phase dispersed in an emulsion, micelles, and/or an internal phase of suspensions). The lipid nanoparticle can be used for transferring the nucleic acid molecule NA or for encapsulation of the nucleic acid molecule NA and/or the peptides or proteins expressed from the nucleic acid molecule NA.

For example, the lipid nanoparticle composition can comprise a ionization lipid, a phospholipid (or helper lipid), a structure-maintaining lipid (or cholesterol lipid) and/or polyethylene glycol (PEG)-lipid.

Formulations containing cationic lipid can be useful for transferring poly-valent anion such as the nucleic acid molecule NA. Other lipid that may be contained in the lipid nanoparticle composition comprises a neutral lipid (i.e., uncharged lipid or zwitterionic lipid), an anionic lipid, the structure-maintaining lipid enhancing transfection of the nucleic acid molecule NA, and a stealth lipid or the PEG-lipid increasing the length of time of the nano particle within a body). Appropriate cationic lipids, neutral lipids, cationic lipids, helper lipids and stealth lipids are disclosed in WO2026/0188840 A1, which is incorporated herein by reference.

For example, the lipid for encapsulation of the nucleic acid molecule NA can be cationic lipids and/or biodegradable lipids. As an example, such lipids can comprise, but is not limited to, (9Z,12Z)-3((4,4-bis(octyloxy)butanoyl)oxy)-2-(((((3-(diethylamino)propoxy)carbonyl)oxy)methyl)propyl-octadeca9,12-dienoate, ((5-((dimethylamino)methyl)-1,3-phenylene)bis(oxy))bis(octane-8,1-diyl)bis(decanoate), 2-((4-(((3-(dimethylamino)propoxy)carbonyl)oxy)hexadecanoyl)oxy)propane-1,3-diyl(9Z,9′Z,12Z,12′Z)-bis(octadeca-9,12-dienoate), 3-(((3-(dimethylamino)propoxy)carbonyl)oxy)-13-(octanoyloxy)tridecyl 3-octylundecanoate and heptatriaconta-6,9,28,31-tetraen-19-yl 4-(dimethylamino)butanoate (also, known as Dlin-MC3-DMA (MC3)).

The phospholipid surrounds and protects core formed by the interaction between the ionization lipid and the active ingredient within the lipid nanoparticles. In addition, the phospholipid binds to the phospholipid bilayers of the target cells and facilitates membrane passages and endosome escape in transferring the active ingredient within the cells. The phospholipid can include neutral, uncharged or zwitterionic phospholipids.

As an example, the phospholipid can comprise, but is not limited to, 5-heptadecylbenzene-1,3-diol (resorcinol), phosphatidylcholine (PLPC), lysophosphatidylcholine, phosphatidylethanolamine (PE), egg phosphatidylcholine (EPC), dioleoylphosphatidylethanolamine (DOPE), distearoylphosphatidylcholine (DSPC), 1,2-distearoyl-sn-glycero-3-phosphocholine (DAPC), palmitoyloleoylphosphatidylcholine (POPC), dioleoylphosphatidylcholine (DOPC), dipalmitoylphosphatidylcholine (DPPC), 1-palmitoyl-2-myristoylphosphatidylcholine (PMPC), dimyristoylphosphatidylcholine (DMPC), 1-Myristoyl-2-palmitoylphosphatidylcholine (MPPC), 1-palmitoyl-2-stearoylphosphatidylcholine (PSPC), 1-stearoyl-2-palmitoylphosphatidylcholine (SPPC), dioleoylphosphatidylglycerol DOPG), dipalmiitoylphosphatidylglycerol (DPPG), distearoylphosphatidylethanolamine (DSPE), dipalmitoylphosphatidylethanolamine, dilauroylphosphatidylcholine (DLPC), 1,2-diarachidoyl-sn-glycero-3-phosphocholine (DBPC), 1,2-dieicosenoyl-sn-glycero-3-phosphocholine (DEPC)), 1,2-dioleoyl-sn-glycero-3-phosphoethanolamine, 1-palmitoyl-2-oleoyl-sn-glycero-3-phosphoethanolamine (POPE), 1-palmitoyl-2-oleoyl-sn-glycero-3-phosphocholine (POPC), 1,2-dioleoyl-sn-glycero-3-[phospho-L-serine](DOPS), 1,2-dioleoyl-sn-glycero-3-[phospho-L-serine] and/or combinations thereof. For example, lipid nanoparticles containing DOPE may be effective for mRNA delivery (excellent drug delivery efficiency for mRNA), and lipid nanoparticles containing DSPE may be effective for siRNA delivery (excellent drug delivery efficiency for siRNA).

The structure-maintaining lipid (cholesterol lipid) can provide rigidity in terms of shape due to lipid filling within lipid nanoparticles (LNPs) and are dispersed on the core and surface of the nanoparticles, improving the stability of the nanoparticles. Additionally, structure-maintaining lipids can enhance transfection of nucleic acid molecules into living cells and/or enhance membrane-fusogenicity.

For example, the structure-maintaining lipid can comprise, but is not limited to, steroids, sterols, and alkyl resorcinols. Alternatively or additionally, the structure-maintaining lipid can comprise, but is not limited to, cholesterols, 5-heptadecyl resorcinol and cholesterol hemi-succinate.

The PEG-lipid contributes to the particle stability of the nanoparticles within the lipid nanoparticles in a serum, and can play a role in preventing aggregation between nanoparticles. For example, the PEG-lipids protect nucleic acid molecules from degrading enzymes when delivering nucleic acid molecules in vivo, enhance the stability of nucleic acids in the body, and can increase the half-life of drugs encapsulated in nanoparticles.

For example, the PEG-lipid can comprise, but is not limited to, polymers with a hydrophilic head such as PEG (polyethylene glycol or polyethylene oxide), poly(oxazoline), poly(vinyl alcohol), poly(glycerol), poly(N-vinyl pyrrolidone), poly-amino acid, and poly-N(2-hydroxypropyl)methacrylamide.

As an example, the PEG lipid an comprise, but is not limited to, PEG-dilauroylglycerol, PEG-dimyristoylglycerol (PEG-DMG, DMG-PEG), PEG-dipalmitoylglycerol, PEG-distearoylglycerol (PEG-DSPE), PEG-dilaurylglycamide, PEG-dimyristylglycamide, PEG-dipalmitoylglycamide, PEG-distearoylglycamide, PEG-cholesterol (1-[8′-(cholest-5-N3[beta]-oxy)carboxamido-3′,6′-dioxaoctanyl]carbamoyl-[omega]-methyl-poly(ethylene glycol), PEG-DMB (3,4-ditetradecoxylbenzyl-[omega]-methyl-poly(ethylene glycol) ether).

The pharmaceutical composition can further comprise the pharmaceutically acceptable carrier. In one embodiment, when the pharmaceutical composition is formulated as liquid, the pharmaceutically acceptable carrier can comprise, but is not limited to, pyrogen-free water; isotonic saline or buffered (water) solution such as phosphate or citrate; plant oil such as peanut oil, cotton seed oil, sesame oil, olive oil, corn oil and cacao fruit oil; glycols such as propylene glycol, glycerol, sorbitol, mannitol and polyethylene glycol; and polyol such as alginic acid. In this case, aqueous buffer including sodium salts, calcium salts, and optionally potassium salts can be used for injecting liquid pharmaceutical composition into bodies. Sodium salts, calcium salts and potassium salts may have halogenized type such as iodine or bromine, hydroxide, carbonate salt, hydrogen carbonate salt or sulfonate salts.

When the pharmaceutical composition is formulated as solid, the pharmaceutically acceptable carrier can comprise solid carrier such as solid filter, liquid filter or diluents, and encapsulating compound may be used as the carrier for administering the composition. For example, the pharmaceutically acceptable carrier may comprise, but are not limited to, sugar such as lactose, glucose and sucrose; starch such as corn starch of potato starch; cellulose or its derivative such as sodium carboxylmethyl cellulose, ethyl cellulose and cellulose acetate; powdered tragacanth; malt; gelatins; tallow; solid lubricant such as stearic acid and magnesium stearate; and calcium sulfate.

The pharmaceutically acceptable carrier can be selected as the administering types of the composition. In one embodiment, the composition may be administered systemically. The administering route can comprise in oral, intracutaneous, intravenous, intra muscular, intra-articular, intrsynovial, intrathecal, intrhepatic, intralesional, intracranial, transdermal, intradermal, intrapumonal, intraperitoneal, intracardial, intraarterial, sublingual topical and/or intranasal.

The pharmaceutical composition may be administered with any convenient type, for example, tablet, powder, capsule, solution, dispersion, suspension, syrup, spray, suppository, gel, emulsion, and patch. For example, the pharmaceutical composition for injection may have a dosage form of sterilized aqueous solution, physiological saline solution and/or mixture thereof. The solution may have adjusted pH of about 7.4.

The pharmaceutically acceptable carrier may comprise hydrogel, adjusted release devices, delayed release devices, polylactic acid and collagen matrix for injection. The pharmaceutically acceptable carrier appropriate for local uses may comprises lotion, cream, gel and similar thereof. If the composition is orally administered, tablet, capsule is preferred unit dosage form.

If necessary, the pharmaceutical composition may further at least one auxiliary substances so as to further increase immunogenicity induced by the pharmaceutically active ingredient and/or the nucleic acid molecule as the adjuvant. For example, substances that allow maturation of dendiric cells (DCs), for example, lipopolysaccharides, TNF-alpha or CD40 ligand, form such auxiliary substances. For example, the auxiliary substance can comprise, but is not limited to, monokines, lymphokines, interleukins or chemokines, that promote the immune response, such as various interleukines, interferons, GM-CSF, G-DSF, M-CSF, LT-beta, TNF-alpha, growth factors such as human growth factor (hGH).

The pharmaceutical composition may additionally contain one or more of buffering agents, stabilizers, surfactants, wetting agents, lubricants, emulsifiers, suspending agents, preservatives, antioxidants, opacifying agents, lubricants, processing aids, colorants, sweeteners, flavoring agents, Diluents, and other known additives that provide an attractive appearance to the drug (i.e., the nucleic acid molecule that is the active ingredient of the invention, gene carrier or vaccine composition) or assist in the manufacture of pharmaceutical products (i.e., medicaments). For example, emulsifiers such as Tween; Wetting agents, for example sodium lauryl sulfate; coloring agent; taste-imparting agents, tablet-forming agents; stabilizer; antioxidant; and conservative.

The contents or the concentration of the nucleic acid molecule NA as the active ingredient is not particularly limited. When the RNA-type nucleic acid NA is used, it is possible to secure safety and stability because the RNA-type nucleic acid molecule NA is dissolved rapidly in the body. In one embodiment, the nucleic acid molecule NA can be used in the pharmaceutical composition with a concentration of about 1 to about 1000 g/ml, for example, about 10 to about 1000 g/ml, but is not limited thereto.

An important factor for an appropriate immune response is the enhancement of different T-cell sub-populations. T-lymphocytes typically differentiate into two subpopulations, that is, T-helper 1 (Th₁) cells and T-helper 2 (Th₂) cells. The subpopulations have immune system that can destroy intracellular (Th₁) pathogens and extracellular (Th₂) pathogens (e.g., antigens). Two Th cell populations differ in the patterns of effector proteins (cytokines) produced by them. In general, Th₁ cells primarily support cellular immune responses through the activation of macrophages and cytotoxic T-cells associated with humoral immunity. On the other hand, Th₂ cells mainly promote humoral immune responses by augmenting B-cells for transformation into plasma cells, associated with cellular immunity, and by the formation of antibodies (e.g., antibodies to antigens). Therefore, the Th₁/Th₂ ratio is quite important in the immune response. Nucleic acid molecule NA enhances both Th₁ and Th₂ immune responses.

As an example, the pharmaceutical composition can be used for preventing tumors and infectious diseases by inducing tumor-specific or pathogen-specific immune responses. Alternatively, the pharmaceutical composition can be used for preventing allergic diseases or disorders and autoimmune diseases, but is not limited thereto.

The pharmaceutical composition can be administered in any convenient dosage form, such as tablets, powders, capsules, solutions, dispersions, suspensions, syrups, sprays, suppositories, gels, emulsions, patches, etc. Such pharmaceutical composition can contain ingredients customary for pharmaceutical preparations, such as diluents, carriers, pH adjusters, sweeteners, bulking agents and further active agents.

The pharmaceutical composition can be prepared in unit dosage form by formulating a pharmaceutically acceptable carrier and/or excipient according to a method that can be easily performed by a person skilled in the art. Alternatively, the pharmaceutical composition can be prepared by placing it in a multi-capacity container. In this case, the formulation may be in the form of a solution, suspension, or emulsion in an oil or aqueous medium, or may be in the form of an extract, powder, granule, tablet, or capsule, and may additionally contain a dispersant or stabilizer.

The formulations or the preparations as used herein can contain two or more active compounds, for example, compounds with complementary activities that do not adversely affect each other, if required for the particular indication to be treated. Alternatively or additionally, the pharmaceutical composition may comprise agents that enhance its function, such as cytotoxic agents, cytokines, chemotherapeutic agents, and grown-inhibiting or growth-enhancing agents. Those molecules are stably present in combination in amounts effective for the intended purposes.

In another embodiment, the gene carrier including the nucleic acid molecule NA can be contained in the pharmaceutical composition. The gene carrier is designed to transport and express the nucleic acid molecule NA encoding one or more therapeutic peptides and/or immunogens. For example, the transcript of the gene of interest can be present in a suitable expression construct to prepare the gene carrier. In one embodiment, the transcript of the gene of interest encoding one or more therapeutic peptides and/or immunogens can be operably linked to the transcription control element TCCE and/or the translation control element TLCE in the expression construct.

For example, the expression construct can be any expression vector in which the nucleic acid molecule NA is inserted. The vector can comprise the nucleic acid molecule NA, and the nucleic acid molecule NA can lined to other nucleotide elements to express a fusion protein or a fusion peptide.

Various techniques known in the art may be used for introducing the gene carrier to the cells. For example, when the gene carrier based upon the viral vectors is fabricated, the gene carrier can be introduced into the host cells by any viral infection methods known in the art. Alternatively, when the gene carrier based upon naked recombinant DNA molecule of plasmid is fabricated, the gene carrier can be transfected into the host cells using anyone of microinjection method, calcium-phosphate precipitate method, electroporation method, liposome-mediated transfection method, DEAE-dextran treating method, and gene bombardment, but is not limited thereto.

### Example 1: Preparation of Nucleic Acid Molecule of RNA Platform

An artificial nucleic acid molecule of RNA platform including Renilla Luciferase encoding sequence in a coding region was prepared. A template DNA having the following ordered sequence was designed:

5′-KpnI recognition site (GGTACC)-T7 promoter (SEQ ID NO: 16)-upstream translation control element derived from human troponin 1 (TNNT1) (SEQ ID NO: 1)-PacI recognition site (TTAATTAA)-Kozak sequence (GCCACC)-Renilla Luciferase encoding sequence (R/L, SEQ ID NO: 15)-ClaI recognition site (ATCGAT)-downstream translation control element derived from TNNT1 (SEQ ID NO: 8)-EcoRI recognition site (GAATTC)-50 adenosines (for tail, A₅₀)-stem I (SEQ ID NO: 17, including GGCCGC, a fragment of reversely complementary to NotI recognition site at 5′ terminus)-50 adenosines (for forming loop, A₅₀)-stem II (SEQ ID NO: 18, including NotI recognition site GCGGCCGC at 3′ terminus)-3′.

The template DNA was cloned to a pGH vector (SEQ ID NO: 19), linearized with a restriction endonuclease, and performed in vitro transcription (IVT) so that the nucleic acid molecule of RNA platform type (hereinafter, “pHJ5C) was prepared.

### Comparative Example 1: Preparation of Nucleic Acid Molecule of RNA Platform

Nucleic acid molecule of RNA platform type (pHJ5L) was prepared as the same process as Example 1, except that the template DNA was designed as follows:

5′-KpnI recognition site (GGTACC)-T7 promoter (SEQ ID NO: 16)-upstream translation control element derived from human troponin 1 (TNNT1) (SEQ ID NO: 1)-PacI recognition site (TTAATTAA)-Kozak sequence (GCCACC)-Renilla Luciferase encoding sequence (R/L, SEQ ID NO: 15)-ClaI recognition site (ATCGAT)-downstream translation control element derived from TNNT1 (SEQ ID NO: 8)-EcoRI recognition site (GAATTC)-first 50 adenosines (for first tail, A₅₀)-linker sequence (SEQ ID NO: 20)-second 50 adenosines (for second tail, A₅₀)-Sap recognition site (GAAGAGC)-Not I recognition site (GCGGCCGC)-3′.

### Comparative Example 2: Preparation of Nucleic Acid Molecule of RNA Platform

Nucleic acid molecule of RNA platform type (pHJ5A) was prepared as the same process as Example 1, except that the template DNA was designed as follows:

5′-KpnI recognition site (GGTACC)-T7 promoter (SEQ ID NO: 16)-upstream translation control element derived from human troponin 1 (TNNT1) (SEQ ID NO: 1)-PacI recognition site (TTAATTAA)-Kozak sequence (GCCACC)-Renilla Luciferase encoding sequence (R/L, SEQ ID NO: 15)-ClaI recognition site (ATCGAT)-downstream translation control element derived from TNNT1 (SEQ ID NO: 8)-EcoRI recognition site (GAATTC)-first 50 adenosines (for first tail, A₅₀)-linker sequence (SEQ ID NO: 20)-second 50 adenosines (for second tail, A₅₀)-third 50 adenosines (for third tail, A₅₀)-Sap recognition site (GAAGAGC)-Not I recognition site (GCGGCCGC)-3′.

### Comparative Example 3: Preparation of Nucleic Acid Molecule of RNA Platform

Nucleic acid molecule of RNA platform type (pHJ5P) was prepared as the same process as Example 1, except that the template DNA was designed as follows:

5′-KpnI recognition site (GGTACC)-T7 promoter (SEQ ID NO: 16)-upstream translation control element derived from human troponin 1 (TNNT1) (SEQ ID NO: 1)-PacI recognition site (TTAATTAA)-Kozak sequence (GCCACC)-Renilla Luciferase encoding sequence (R/L, SEQ ID NO: 15)-ClaI recognition site (ATCGAT)-downstream translation control element derived from TNNT1 (SEQ ID NO: 8)-EcoRI recognition site (GAATTC)-first 50 adenosines (for first tail, A₅₀)-stem-loop element (SEQ ID NO: 21)-second 50 adenosines (for second tail, A₅₀)-Not I recognition site (GCGGCCGC)-3′.

### Comparative Example 4: Preparation of Nucleic Acid Molecule of RNA Platform

Nucleic acid molecule of RNA platform type (pHJ5PP) was prepared as the same process as Example 1, except that the template DNA was designed as follows:

5′-KpnI recognition site (GGTACC)-T7 promoter (SEQ ID NO: 16)-upstream translation control element derived from human troponin 1 (TNNT1) (SEQ ID NO: 1)-PacI recognition site (TTAATTAA)-Kozak sequence (GCCACC)-Renilla Luciferase encoding sequence (R/L, SEQ ID NO: 15)-ClaI recognition site (ATCGAT)-downstream translation control element derived from TNNT1 (SEQ ID NO: 8)-EcoRI recognition site (GAATTC)-first 50 adenosines (for first tail, A₅₀)-first stem-loop element (SEQ ID NO: 21)-second 50 adenosines (for second tail, A₅₀)-second stem-loop element (SEQ ID NO: 22, including NotI recognition site GCGGCCGC at 3′ terminus)-3′

### Experimental Example 1: In Vitro Expression

Nor10 cells of mouse muscle cell line were inoculated to a 48-well plate with a concentration of 8×10⁴ cells/well. The plate was put into a 37° C. incubator to allow the cells to attach to the plate and grow for 24 hours. Each of the mRNAs prepared in Comparative Examples 1, 2 and 4 and Example 1 was transfected to the cell line with a concentration of 500 ng/well. Lipofectamine 2000™ was used at 1 μl per 1 μg of each mRNA and treated in an OPTI-MEM medium for more than 30 minutes to ensure sufficient mixing. When the mRNA and Lipofectamine 2000™ were mixed, the 48-well plate with the attached cells was taken out, the supernatant was removed, washed with PBS, and the supernatant was removed again. 100 μl of medium was added on the cells, and 100 μl of a mixture of mRNA and Lipofectamine 2000™ was added.

At this time, only 200 μl medium was added to the negative control group. The plate was placed back in the 37° C. incubator and was taken out at 6 and 24 hours later to confirm expression. When 6 and 24 hours have elapsed, the plate was taken out, all supernatant was removed, the plate was washed once with PBS, and 80 μl of Renilla lysis buffer from the Renilla Luciferase assay system kit (Promega) was added on the cells to sufficiently destroy the cells. After doing so, the expression level of Renilla Luciferase was measured by spectrophotometer according to the kit instructions.

Each of FIGS. 5 and 6 illustrates result of measuring relative expression efficiency where the expression efficiency transfected with other nucleic acid molecules was compared to the expression level of Renilla Luciferase in the Nor10 cell line transfected with the mRNA prepared in Comparative Example 1 was set to 1. The nucleic acid molecule pHJ5C prepared in Example 1 showed excellent expression efficiency, that is, showed still excellent expression even after 24 hours elapsed.

### Example 2: Preparation of Nucleic Acid Molecule of RNA Platform

An artificial nucleic acid molecule of RNA platform type (pHJ5C-FC) was prepared as the same process as Example 1, except that the human TNNT1 downstream translation control element was modified to two downstream translation control elements comprising a first downstream translation control element derived from human RPS27 (SEQ ID NO: 13) and a second downstream translation control element derived from human FTL (SEQ ID NP: 10).

### Comparative Example 5: Preparation of Nucleic Acid Molecule of RNA Platform

An artificial nucleic acid molecule of RNA platform type (pHJ5L-FC) was prepared as the same process as Comparative Example 1, except that the human TNNT1 downstream translation control element was modified to two downstream translation control elements comprising a first downstream translation control element derived from human RPS27 (SEQ ID NO: 13) and a second downstream translation control element derived from human FTL (SEQ ID NP: 10).

### Comparative Example 6: Preparation of Nucleic Acid Molecule of RNA Platform

An artificial nucleic acid molecule of RNA platform type (pHJ5P-FC) was prepared as the same process as Comparative Example 2, except that the human TNNT1 downstream translation control element was modified to two downstream translation control elements comprising a first downstream translation control element derived from human RPS27 (SEQ ID NO: 13) and a second downstream translation control element derived from human FTL (SEQ ID NP: 10).

### Example 3: Preparation of Nucleic Acid Molecule of RNA Platform

An artificial nucleic acid molecule of RNA platform type (pHJ5C-GF) was prepared as the same process as Example 1, except that the human TNNT1 downstream translation control element was modified to two downstream translation control elements comprising a first downstream translation control element derived from DEFA5 (SEQ ID NO: 14) and a second downstream translation control element derived from human RPS27 (SEQ ID NP: 13)

### Comparative Example 7: Preparation of Nucleic Acid Molecule of RNA Platform

An artificial nucleic acid molecule of RNA platform type (pHJ5C-GF) was prepared as the same process as Comparative Example 1, except that the human TNNT1 downstream translation control element was modified to two downstream translation control elements comprising a first downstream translation control element derived from DEFA5 (SEQ ID NO: 14) and a second downstream translation control element derived from human RPS27 (SEQ ID NP: 13).

### Comparative Example 8: Preparation of Nucleic Acid Molecule of RNA Platform

An artificial nucleic acid molecule of RNA platform type (pHJ5P-GF) was prepared as the same process as Example 2, except that the human TNNT1 downstream translation control element was modified to two downstream translation control elements comprising a first downstream translation control element derived from DEFA5 (SEQ ID NO: 14) and a second downstream translation control element derived from human RPS27 (SEQ ID NP: 13).

### Experimental Example 2: In Vitro Expression

Nor10 cells of mouse muscle cell line and HeLa cells of human cell line were inoculated to a 48-well plate with a concentration of 8×10⁴ cells/well, respectively. The plate was put into a 37° C. incubator to allow the cells to attach to the plate and grow for 24 hours. Each of the mRNAs prepared in Comparative Examples 1, 3, 5 to 8 and Examples 1-3 was transfected to the cell line with a concentration of 500 ng/well. Then, expression levels of Renilla Luciferase were measured with the same process as Experimental Example 1. Each of FIGS. 7 to 11 illustrates result of measuring relative expression efficiency where the expression efficiency transfected with other nucleic acid molecules was compared to the expression level of Renilla Luciferase in the Nor10 cell line transfected with the mRNA prepared in Comparative Example 1 was set to 1. The nucleic acid molecules prepared in Examples 1-3 showed excellent expression efficiency, that is, showed still excellent expression even after 24 hours elapsed.

### Example 4: Preparation of Nucleic Acid Molecule of RNA Platform

An artificial nucleic acid molecule of RNA platform type was prepared as the same process as Example 1, except modified polynucleotides encoding peptides of E6 region and E6 region of HPV was inserted to the coding region. The template DNA was designed as follows:

5′-KpnI recognition site (GGTACC)-T7 promoter (SEQ ID NO: 16)-upstream translation control element derived from human troponin 1 (TNNT1) (SEQ ID NO: 1)-PacI recognition site (TTAATTAA)-Kozak sequence (GCCACC)-HPV type 18 E7 derived nucleotides (SEQ ID NO: 24)-GSGSG linker (SEQ ID NO: 32)-HPV type 16 E7 derived nucleotides (SEQ ID NO: 25)-GSGSG linker (SEQ ID NO: 32)-HPV type 16 E6 derived nucleotides (SEQ ID NO: 26)-GSGSG linker (SEQ ID NO: 32)-HPV type 18 E6 derived nucleotides (SEQ ID NO: 29, SEQ ID NO: 30, and SEQ ID NO: 31)-ClaI recognition site (ATCGAT)-downstream translation control element derived from TNNT1 (SEQ ID NO: 8)-EcoRI recognition site (GAATTC)-50 adenosines (for tail, A₅₀)-stem I (SEQ ID NO: 17, including GGCCGC, a fragment of reversely complementary to NotI recognition site at 5′ terminus)-50 adenosines (for forming loop, A₅₀)-stem II (SEQ ID NO: 18, including NotI recognition site GCGGCCGC at 3′ terminus)-3′.

The template DNA was cloned to a pGH vector (SEQ ID NO: 19), linearized with a restriction endonuclease, and performed in vitro transcription (IVT) so that the nucleic acid molecule of RNA platform type was prepared.

### Experimental Example 3: In Vitro Expression

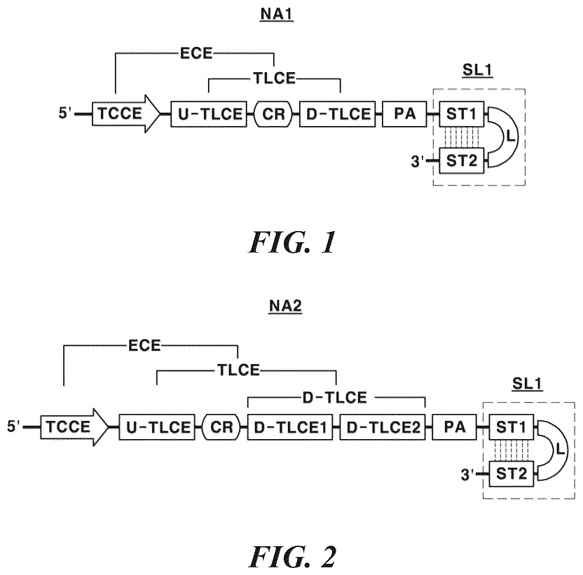

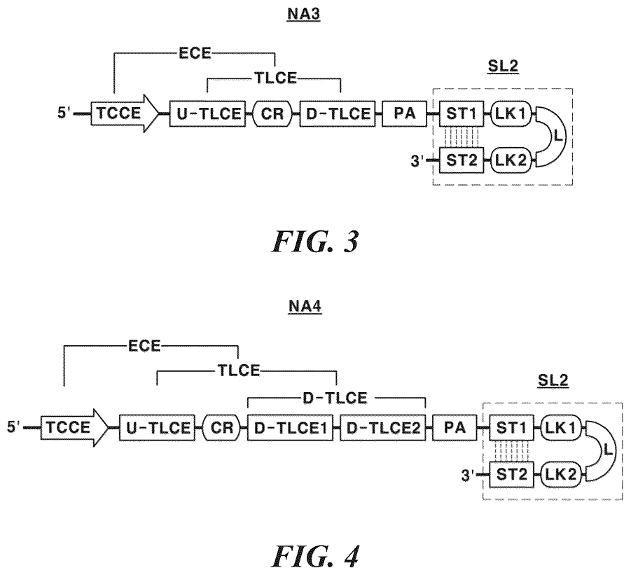

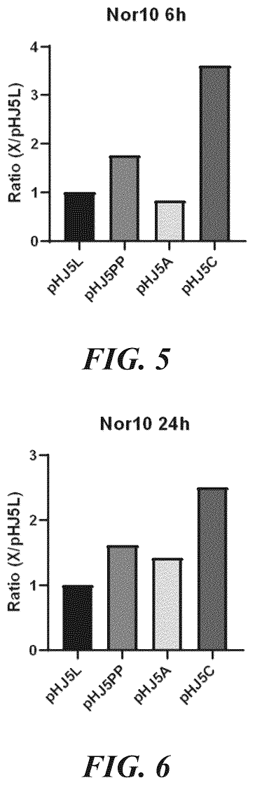

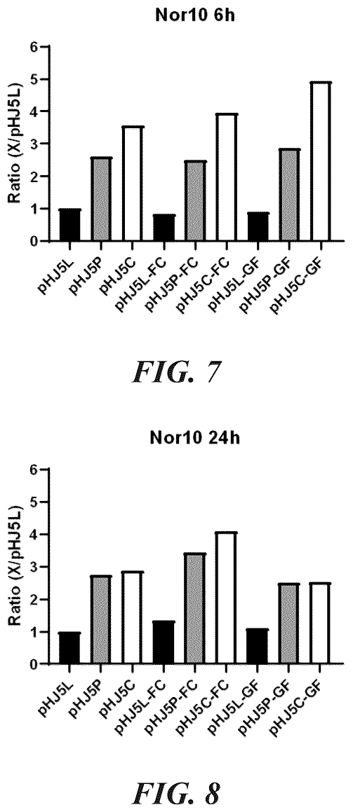

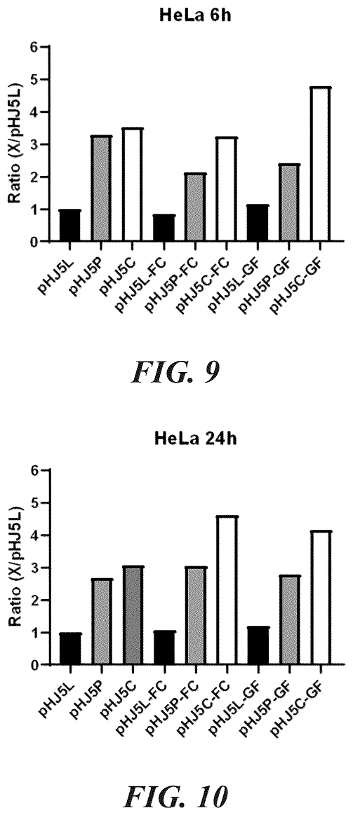

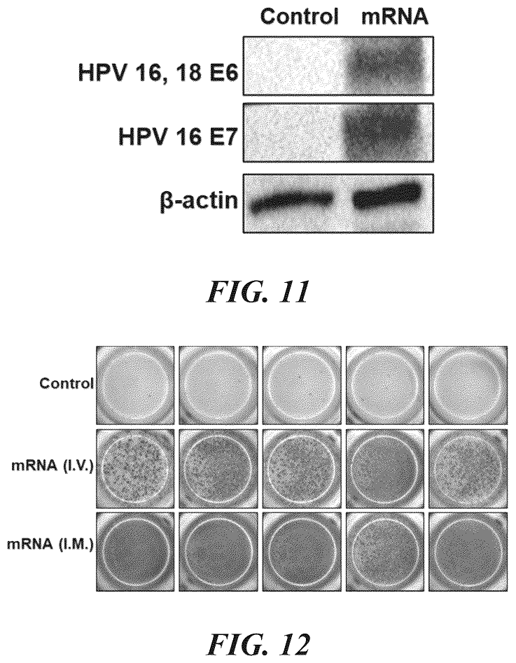

The cell line was transfected with the same process as Experimental Example 1, except the nucleic acid molecule prepared in Example 4 was inoculated to Nor10 cell lines. Proteins in the cell line were extracted using Lipa solution, a type of surfactant. After protein electrophoresis, the protein was electrophoretically transferred to a nitrocellulose membrane. Then, Western-blotting was performed using antibodies that bind to the E6 and E7 proteins of HPV types 16 and 18 to confirm the expression of the protein inserted into the coding region of the nucleic acid molecule. As illustrated in FIG. 11, it was confirmed that the protein inserted into the nucleic acid molecule of Example 4 was expressed normally.

### Experimental Example 4: Measurement of Immune Response

The nucleic acid molecule prepared in Example 4 was injected into mice, and the immune response was evaluated. Nucleic acid molecule was injected intravenously (I.V.) or intramuscularly (I.M.) into mice, respectively, and 40 μg and 20 μg of nucleic acid molecule were inoculated at one-week intervals. One week after the last vaccination, the mice were necropsied, spleens were isolated, and immune responses were analyzed. As a negative control, saline, not nucleic acid molecules, was inoculated.

An antibody that binds to IFN-gamma was bound to the bottom of a 96-well plate, and spleen cells from autopsied mice were cultured on the antibody with a concentration of 5×10⁵ cells/well using two wells per individual. Nutrient medium was added to one of the two wells, and nutrient medium containing HPV-derived peptides was supplied to the other well, and then ELISPOT assay was performed. As illustrated in FIG. 12, the secretion of IFN-gamma significantly increased in the spleen of the group inoculated with the nucleic acid molecule into which the HPV-derived polynucleotide was inserted into the well supplied with the nutrient medium containing the HPV-derived peptide.

In addition, spleen cells from autopsied mice were cultured in a 96-well plate with a concentration of 1×10⁶ cells/well using two wells per individual. Nutrient medium was added to one of the two wells, and nutrient medium containing HPV-derived peptides was supplied to the other well, followed by flow cytometry assay. As illustrated in FIG. 13, among the spleen cells of the group inoculated with nucleic acid molecule in wells supplied with nutrient medium containing HPV-derived peptides, the proportion of CD8 IFN-gamma positive T cells was also high. In particular, a high immune response was induced in the spleen of mice injected intramuscularly with nucleic acid molecules.

### Experimental Example 5: Assay of Therapeutic Effect of HPV Derived Disease

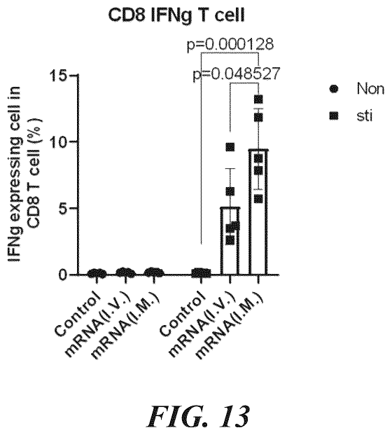

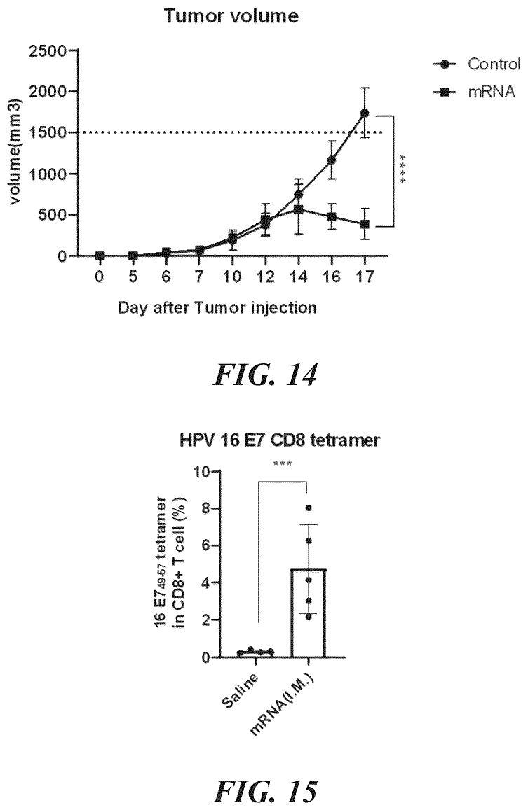

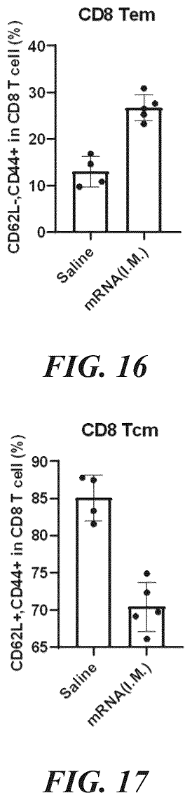

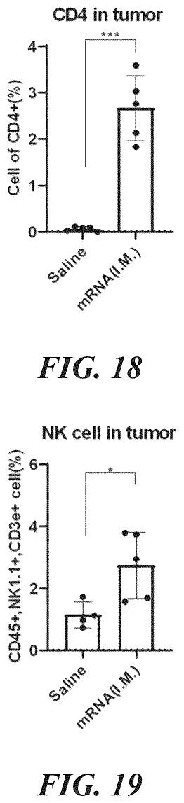

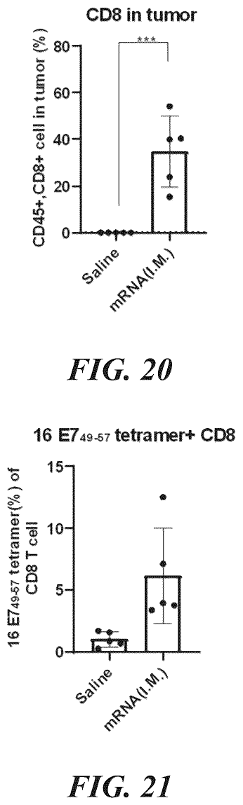

The TC-1 tumor cell line was injected into mice, and 20 μg of the composition formulated with LNP (lipid nano particles) of the nucleic acid molecule prepared in Example 4 was intramuscularly injected 6, 11, and 17 days after tumor cell injection, respectively. Saline was injected as a negative control. Six days after the last inoculation, the mice were autopsied, spleen was isolated, and the size of tumor cells was measured. As illustrated in FIG. 14, the tumor volume was significantly reduced compared to the negative control.

Mice spleens were isolated and cultured in a 96-well plate with a concentration of 1×10⁶ cells/well. CD8 tetramer specific to the E7 region of HPV-type 16 and a fluorescent antibody that binds to immune T cell surface factors were combined and flow cytometry was performed to assay the immune responses. As illustrated in FIGS. 15 to 17, an increase in CD8 cells specific to the E7 region of HPV-type 16 was revealed through tetramer staining. In addition, it was confirmed that CD8 memory T cells were highly induced and that inoculation with the nucleic acid molecule prepared in Example 4 could contribute to preventing subsequent cancer recurrence.

The tumor tissue identified above was separated, the separate tumor tissue was treated with Dnase1 and Hyaluronidase to obtain tumor cells, and 5×10⁵ cells/well of the tumor cells was cultured in in a 96-well plate. The tumor cells were treated with fluorescent antibody that binds bind to CD4, CD8, and NK surface proteins and CD8 tetramer specific to the E7 peptide of HPV-type 16, and assayed by flow cytometry. The assay results are illustrated in FIGS. 18 to 21. It was confirmed that all immune cells, including CD4, CD8, and NK cells, especially CD8 T cells specific to the E7 peptide of HPV-type 16 had infiltrated to the cells.

While the present disclosure has been described with reference to exemplary embodiments and examples, these embodiments and examples are not intended to limit the scope of the present disclosure. Rather, it will be apparent to those skilled in the art that various modifications and variations can be made in the present disclosure without departing from the spirit or scope of the invention. Thus, it is intended that the present disclosure covers the modifications and variations of the present disclosure provided they come within the scope of the appended claims and their equivalents.

What is claimed is:

1. A nucleic acid molecule comprising:
a coding region encoding one or more peptides or fragments thereof; and
an element located adjacently to the coding region and having a stem-loop structure,
wherein a loop in the stem-loop structure comprises a single type of nucleotide.

2. The nucleic acid molecule of claim 1, wherein the nucleic acid molecule further comprises at least one expression control element operably linked to the coding region.

3. The nucleic acid molecule of claim 2, wherein the at least one expression control element comprises at least one transcription control element and at least one translation control element.

4. The nucleic acid molecule of claim 3, wherein the at least one translation control element comprises an upstream translation control element located upstream of the coding region and a downstream translation control element located downstream of the coding region.

5. The nucleic acid molecule of claim 4, wherein the downstream translation control element comprises a first downstream translation control element and a second downstream translation control element located sequentially and downstream of the coding region.

6. The nucleic acid molecule of claim 3, wherein the at least one translation control element comprises an element having a cap-dependent translation initiation activity.

7. The nucleic acid molecule of claim 3, wherein the at least one translation control element comprises a translation control element having an Internal Ribosomal Entry Site (IRES) activity.

8. The nucleic acid molecule of claim 1, wherein the nucleic acid molecule further comprises a polyadenylation signal sequence or a polyadenosine sequence located between the coding region and the element having the stem-loop structure, or located downstream of the element having the stem-loop structure.

9. The nucleic acid molecule of claim 1, wherein the nucleic acid molecule has an RNA type.

10. The nucleic acid molecule of claim 1, wherein the loop consists of adenosines or analogs thereof, or nucleotides transcribed thereto.

11. The nucleic acid molecule of claim 1, wherein a stem element located downstream of the element having the stem-loop structure comprises at least one restriction recognition site or a fragment thereof.

12. The nucleic acid molecule of claim 1, wherein the element having the stem-loop structure further comprises a linker element interposed between a stem and the loop.

13. The nucleic acid molecule of claim 1, wherein the coding region encodes one or more therapeutic proteins or peptides.

14. The nucleic acid molecule of claim 1, wherein the coding region encodes one or more peptides derived from human papilloma virus (HPV), a fragment thereof, or a tumor suppressive or attenuation peptide thereof.

15. The nucleic acid molecule of claim 14, wherein the coding region encodes at least one of E6 peptide and E7 peptide of the human papilloma virus, a fragment thereof, or a tumor suppressive peptide thereof.

16. The nucleic acid molecule of claim 14, wherein the coding region encodes one or more peptides derived from at least one human papilloma virus (HPV) type of an HPV-16 type or an HPV-18 type, a fragment thereof, or a tumor suppressive or attenuation peptide thereof.

17. The nucleic acid molecule of claim 1, wherein the coding region encodes one or more reporter peptides, one or more marker or selection peptides, or a fragment thereof.

18. The nucleic acid molecule of claim 1, wherein the coding region encodes one or more immunogens, or a fragment thereof.

19. A recombinant expression vector including the nucleic acid molecule of claim 1.

20. A method of treating or preventing a disease in a subject, the method comprising:
administering a therapeutically effective amount of the nucleic acid molecule of claim 1 or an expression construct including the nucleic acid molecule to the subject.
